(19) United States
(12) Patent Application Publication
(10) Pub. No.: US 2009/0182011 A1
(43) Pub. Date: Jul. 16, 2009

(54) Chimeric Nitrate Esters and Use of the Same in a Treatment for Depression

(75) Inventor: Gregory R.J. Thatcher, Chicago, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: UNIVERSITY OF ILLINOIS CHICAGO, Chicago, IL (US)

(21) Appl. No.: 12/354,418

(22) Filed: Jan. 15, 2009

Related U.S. Application Data
(60) Provisional application No. 61/021,426, filed on Jan. 16, 2008.

(51) Int. Cl. A61K 31/4436; A61K 31/4525; A61K 31/4535; A61K 31/451; C07D 409/04; C07C 203/00; C07D 405/12; A61P 25/24

(52) U.S. Cl. 514/321; 514/324; 514/330; 514/337; 514/487; 546/202; 546/281.1; 558/482; 546/197

(57) ABSTRACT

Chimeric nitrate esters and their use in the treatment of depression are disclosed. The chimeric nitrate esters also are useful in the treatment of depression and comorbidity associated with aging.

### CROSS REFERENCE TO RELATED APPLICATION

This application claims the benefit of U.S. provisional patent application 61/021,426, filed Jan. 16, 2008, incorporated herein by reference.

### STATEMENT OF GOVERNMENTAL INTEREST

This invention was made with U.S. government support under grant number AT002299 awarded by the National Institutes of Health (NIH). The U.S. government has certain rights in the invention.

### FIELD OF THE INVENTION

The present invention relates to chimeric nitrate esters useful in the treatment of depression. More particularly, the present invention relates to nitrate esters of antidepressant drugs, such as nitrate esters of selective serotonin reuptake inhibitors (SSRIs), that provide both an antidepressant drug, e.g., SSRI, and a nitric oxide (NO) mimetic in vivo. The nitrate esters also are useful in treating diseases of the aged wherein depression and dementia are comorbid.

### BACKGROUND OF THE INVENTION

Depression affects 17% of the overall population, with prevalence twice as high in women. Despite nearly 50 years of relatively effective treatment for depression, currently used antidepressant drugs still exhibit many of the same disadvantages. For example, current antidepressant drugs suffer from: (a) a wide variety of adverse side effects, from headaches to tremors, that adversely impact patient compliance; (b) a therapeutic lag period, e.g., 2-6 weeks of treatment are required before antidepressant effects are fully developed; and (c) limited effectiveness, e.g., only about 70% of depressions respond to tricyclic antidepressants or SSRIs. Total US sales of antidepressant drugs in 2004, dominated by SSRIs, amounted to about $11 billion. The SSRI fluoxetine, which was introduced in 1985, still accounts for about 16% of total antidepressant scripts.

Many major pharmaceutical companies are actively pursuing next generation antidepressants, many of which are designed to supplement SSRI activity by an action on different receptors or transporters, including attempts to negate the “therapeutic lag” period. Little of this activity is directed to the treatment of depression associated with aging and neurodegenerative disorders.

Aging, dementia, and depression are correlated. Dementia affects about 6% of individuals at 65 years, and about 24% of individuals over 85 years. “Late onset depression” is a descriptive term for depression first diagnosed after the age of 65 years. Late onset depression shares many characteristics with earlier onset depression, but some distinctions include greater apathy, less personality dysfunction, and more prominent cognitive deficits, including more impaired memory and executive functioning. Insomnia and sleep disturbance are major symptoms of depression in old age, although this may reflect increasing sleep disturbance in older people generally. Comorbid depression in dementia patients is associated with increased disability, more functional and behavioral problems, and greater stress to caregivers.

A significant aspect of depression in older, cognitively impaired persons is a high mortality rate. A number of studies indicate a reduced survival rate in patients suffering from both dementia and depression compared to patients suffering from either syndrome separately. For example, one study found that, by year three, 60% of individuals diagnosed with dementia and mild depression had died or developed a chronic mental illness.

A wide range of theories have been postulated to account for the comorbidity of depression and dementia. Two primary theories exist: (a) a common genetic, neuropathological, or neurochemical basis which favors the development of both disorders and (b) neuropathological changes resulting from depression that cause an increased vulnerability or sensitivity to other risk factors for dementia. A consistent finding is that depression more than ten years prior to onset of dementia is late onset dementia, which suggests that depression is not a mere prodromal symptom of dementia. Other studies found an association between depression and subsequent dementia. However, one study suggested that the depressive symptoms are actually an early manifestation, rather than predictor, of dementia.

Recent research has correlated depression and dementia to anatomical and chemical changes occurring in the brain with age. Degeneration of the cereleus locus and substantia nigra is consistent anatomical changes common to individuals suffering both dementia and depression, and is demonstrable over other hallmarks of dementia alone, including senile plaques and neurofibrillary tangles. This finding indicates that depression associated with dementia is related to a catecholaminergic deficit caused by a degeneration of brain areas involved with catecholamine production. Importantly, dementia sufferers with degeneration of both nuclei were more likely to suffer from depression than sufferers with degeneration of only one nucleus. Conversely, a theory exists suggesting that a loss of serotonin producing neurons occurs with ventricular enlargement found in dementia and depression.

A long held theory is that depression causes changes to the hypothalamic-pituitary-adrenal axis, which in turn causes alterations to neurotransmitter levels predisposing those with depression to develop dementia. Depression increases cortisol levels, thus causing damage to the hypothalamus, further impairing the endocrine axis and causing degeneration to the hippocampus and medial temporal lobe, which may be associated with the cognitive symptoms of dementia.

Regardless of the mechanisms of onset and progression, dementia and depression are common in older populations. Treatment of depressive syndromes of dementia, of syndromes that markedly increase the morbidity associated with dementia, and of the cognitive deficits associated with dementia represent unmet needs. The present invention is directed to this need and to treating depression in all age groups, and to substantially reducing the “therapeutic lag” period associated with present day antidepressant drugs.

### SUMMARY OF THE INVENTION

The present invention is directed to chimeric nitrate esters useful in the treatment of depression. More particularly, the present invention is directed to chimeric nitrate esters of antidepressant drugs, such as chimeric nitrate esters of SSRIs, that provide both an antidepressant drug, e.g., an SSRI, and an NO mimetic in vivo.

Therefore, one aspect of the present invention is to provide chimeric nitrate esters useful to treat individuals suffering from depression. An important aspect of the present invention is to provide chimeric nitrate esters of antidepressant drugs, wherein the therapeutic lag period of the antidepressant drug is substantially reduced, e.g., from 2-6 weeks to 2-7 days. Another aspect of the present invention is to provide chimeric nitrate esters useful in the treatment of neuorodegenerative disorders where depression is a comorbidity, symptom, or prodromus of the disorder, e.g., late onset dementia.

Still another aspect of the present invention is to provide chimeric nitrate esters of compounds that act as an SSRI. In another embodiment, the chimeric nitrate ester is a nitrate ester of modafinil. The chimeric nitrate esters act to provide both an antidepressant drug and an NO mimetic that can act as an NO donor. The present chimeric nitrate esters therefore are metabolized to provide an antidepressant drug and an NO donor for the treatment of depression.

In one aspect, a present nitrate ester has a structural formula (I):

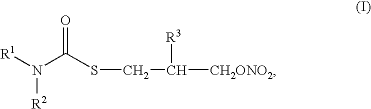

wherein R¹ is selected from the group consisting of C₁₋₆alkyl, C₃₋₈cycloalkyl, C₃₋₈heterocycloalkyl, aryl, and heteroaryl;

R² is selected from the group consisting of C₁₋₆alkyl, C₃₋₈cycloalkyl, C₃₋₈heterocycloalkyl, aryl, and heteroaryl, and or

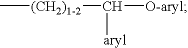

R¹ and R² are taken with the nitrogen atom to which they are attached to form a 5- or 6-membered ring, said ring substituted with an aryl or heteroaryl group and optionally with —(CH₂)_{1,2}—O-aryl; and

R³ is H or ONO₂,

or a pharmaceutically acceptable salt, prodrug, hydrate, or solvate thereof.

In another aspect, a present nitrate ester has a structural formula (II):

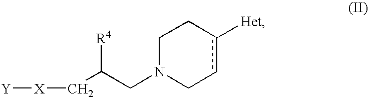

wherein X is null, O, or S;

R⁴ is —OH or —ONO₂;

Y is —CH₂aryl; —(CH₂)₁₋₂ONO₂, or

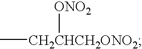

Het is heteroaryl; and

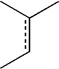

is a carbon-carbon single bond or a carbon-carbon double bond,

or a pharmaceutically acceptable salt, prodrug, hydrate, or solvate thereof.

In yet another aspect, a present nitrate ester has a structural formula (III):

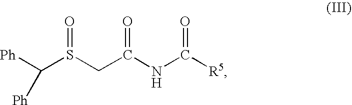

wherein Ph is phenyl; and

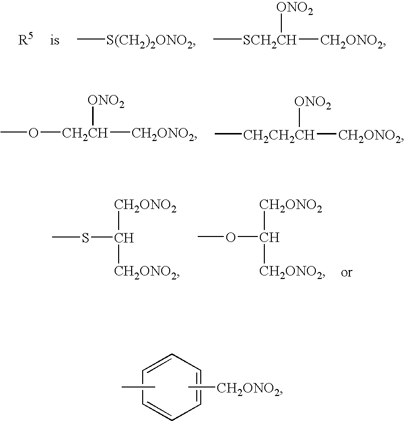

or a pharmaceutically acceptable salt, prodrug, hydrate, or solvate thereof.

In one embodiment, the present invention provides a method of treating depression by administering a therapeutically effective amount of a chimeric nitrate ester of an antidepressant drug, for example a compound of structural formula (I), (II), or (III), to an individual in need thereof. In preferred embodiments, the therapeutic lag period is reduced from a number of weeks to 2 to 7 days.

Another embodiment of the present invention is to provide a composition useful in treating depression comprising (a) a chimeric nitrate ester of an antidepressant drug, such as a nitrate ester of structural formula (I), (II), or (III), and (b) an excipient and/or pharmaceutically acceptable carrier.

In a further embodiment, the invention provides for use of a composition comprising a chimeric nitrate ester of an antidepressant drug, such as a nitrate ester of structural formula (I), (II), or (III), for the manufacture of a medicament for treating depression.

Still another embodiment of the present invention is to provide a method of treating a neurodegenative disorder wherein depression is a comorbidity, symptom, or prodromus, such as late onset depression associated with dementia. The method also alleviates cognitive deficits associated with depression.

In yet another embodiment of the present invention, a method is provided for manufacturing a chimeric nitrate ester of an antidepressant drug comprising condensing an amine-containing compound with an activated carbonyl compound, e.g., phosgene, carbonyl diimidazole, or triphosgene, and an appropriate mercaptan, such that a chimeric nitrate ester is formed, e.g., of structural formula (I), (II), or (III). In one aspect of this embodiment, the chimeric nitrate ester has a thiocarbamate group that acts as a labile linker between the antidepressant drug and the nitrate ester of the chimeric agent.

In a further embodiment, a method is provided for manufacturing a chimeric nitrate ester of structural formula (I) comprising reacting a thiocarbamate derivative of an antidepressant drug with a brominating agent, including, but not limited to, carbon tetrabromide, 1,3-dibromo-5,5-dimethylhydantoin, N-bromosuccinimide, and hydrobromic acid, to produce an alkyl bromide, then nitrating the resulting bromide, for example, with silver nitrate.

These and other aspects and features of the present invention will become apparent from the following detailed description of the preferred embodiments.

### BRIEF DESCRIPTION OF THE DRAWINGS

FIG. 1 is a plot of % Fluoxetine/Total vs. Time (min) showing the bioactivation of NO-fluoxetine in buffer solutions of pH 7.0, 7.4, and 8.0 (50 mM phosphate buffer solution);

FIG. 2 contains bar graphs of Percent Inhibition (%) vs. 100 μM of fluoxetine, Linker-fluoxetine, and NO-fluoxetine in the in vitro serotonin reuptake inhibition assay;

FIG. 3 contains bar graphs of Five Second Blocks in the modified forced swimming test for struggle, swimming, and immobility for NO-fluoxetine and a control;

FIG. 4 contains bar graphs of 48 hour Memory Retention Latency for a Negative Control, Positive Control, Fluoxetine, and NO-Fluoxetine;

FIG. 5 contains bar graphs for Percent Inhibition (%) vs. 100 nM of Linker-SSRI and NO-SSRI for three SSRIs in the in vitro serotonin reuptake inhibition assay.

### DETAILED DESCRIPTION OF THE PREFERRED EMBODIMENTS

The present invention is described in connection with preferred embodiments. However, it should be appreciated that the invention is not limited to the disclosed embodiments. It is understood that, given the description of the embodiments of the invention herein, various modifications can be made by a person skilled in the art. Such modifications are encompassed by the claims below.

As used herein, the term “chimeric nitrate ester” means an organic nitrate compound containing an antidepressant pharmacophore and an NO mimetic. Synonymously, the term “nitrate ester of an antidepressant” means a compound, either known or unknown, that exhibits efficacy in treating various forms and/or symptoms of depression, and that has been modified into the form of a nitrate ester, i.e., having one or more —ONO₂ moiety. Typically, the —ONO₂ moiety is bound to the antidepressant compound via a linker moiety, and preferably via a thiocarbamate linkage (—N—C(═O)—S—), carbamate linkage (—N—C(═O)—S—), or amide linkage (—N—C(═O)—CH₂—). In other preferred embodiments, the nitrate ester of an antidepressant has the —ONO₂ moiety positioned (β, γ, or δ) to the sulfur atom of the thiocarbamate linkage. The nitrate ester of an antidepressant, after administration, is metabolized to provide the antidepressant compound and an NO mimetic. For example, a nitrated fluoxetine, a nitrated paroxetine, and a nitrated modafinil are chimeric nitrate esters and nitrate esters of an antidepressant of the present invention. A chimeric nitrate ester can contain one or more, and typical one or two, nitrate moiety.

As used herein, the terms “treat,” “treating,” “treatment,” and the like refer to eliminating, reducing, or ameliorating a disease or condition and/or a symptom associated therewith. Although not precluded, treating a disease or condition does not require that the disease, condition, or symptom associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a chimeric nitrate ester to an individual in need of such treatment.

Within the meaning of the invention, “treatment” also includes relapse prophylaxis or phase prophylaxis, as well as the treatment of acute or chronic signs, symptoms, and/or malfunctions. The treatment can be orientated symptomatically, for example, to suppress symptoms. It can be effected over a short period, be oriented over a medium term, or can be a long-term treatment, for example within the context of a maintenance therapy.

The term “therapeutically effective amount” or “effective dose” as used herein refers to an amount of a chimeric nitrate ester that is sufficient, when administered by a method of the invention, to efficaciously deliver a chimeric nitrate ester for the treatment of condition or disease of interest to an individual in need thereof. In the case of depression, the therapeutically effective amount of a present chimeric nitrate ester treats apathy, personality dysfunction, cognitive deficits, impaired memory, insomnia, and sleep disturbances, for example, preferably with a substantially shortened therapeutic lag period.

The term “container” means any receptacle and closure therefor suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

The use of the terms “a”, “an”, “the”, and similar referents in the context of describing the invention (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the invention and is not a limitation on the scope of the invention unless otherwise claimed. No language in the specification should be construed as indicating any non-claimed element as essential to the practice of the invention.

Nitric oxide (NO) signaling is essential for normal physiological function in the central nervous system (CNS), and is compromised in many disease states. NO can serve as a retrograde synaptic messenger, as an intracellular messenger, and as a lateral diffusible messenger in the CNS. NO plays a critical role in signal transduction cascades that are compromised in dementia, and thereby contribute to the symptoms of cognitive impairment that characterize Alzheimer's Disease (AD). NO activates soluble guanylyl cyclase (sGC) to release cyclic guanosine-2′:3′-monophosphate (cGMP). NO/cGMP signaling is important for modulating synaptic transmission and plasticity in brain regions, such as the hippocampus and cerebral cortex, which are critical for learning and memory (H. Son et al., Learn Mem 1998, 5, 231-245; and Y. F. Lu et al., J Neurosci 1999, 19, 10250-10261). Evidence exists that NO may positively impact learning, memory, and cognition through cGMP-dependent and independent pathways (T. M. Edwards et al., Neurobiol Learn Mem 2002, 77, 313-326).

Compounds that mimic the effects of NO, i.e., NO mimetics, bypass cholinergic receptor activation and are expected to provide multiple pathways of treating and circumventing dementia. The action of an appropriate nitrate in the hippocampus modulates signaling cascades, in part via MAPK/ERK (mitogen activated protein kinase-extracellular signal-regulated kinase) and CREB (cAMP responsive element binding protein), leading to amelioration of learning and memory pathways under pathophysiological conditions which might lead to neurodegeneration.

Nitrates are NO mimetics that can act as NO donors. In contrast to other NO donors, nitrates do not release high fluxes of NO, which is potentially harmful. Bioactivation and metabolism of nitrates is a variable that strongly influences activity and pharmaceutical use. Whereas the potent hypotensive effects of the organic nitrate vasodilator nitroglycerin may be deleterious in the treatment of depression and dementia, it is theorized herein, but not relied upon, that regulation of systemic versus central effects is required for development of new and useful therapeutic agents useful in the treatment of depression, dementia, and comorbidity associated with aging.

In accordance with the present invention, the central action of a present chimeric nitrate ester is procognitive, that is, triggering signaling pathways leading to reversal of cognitive deficits associated with neurodegeneration and aging. In addition, a present chimeric nitrate ester reinforces production of trophic factors associated with antidepressant activity and neurogenesis. Because nitrate esters show precognitive and antidepressant activity, the regulation of these two effects is achieved through utilization of a present chimeric nitrate ester, and this activity is enhanced by structural incorporation of a chemical moiety having antidepressant activity, e.g., an SSRI or modafinil.

Thus, the present invention is directed to synthetic organic nitrate esters incorporating antidepressant activity as new and useful therapeutic agents for treatment of depression, dementia, and comorbidity associated with aging. It will be appreciated, therefore, that the chimeric nitrate esters can be used for treatment of depression, including depression associated with other psychiatric disorders linked with cognitive deficits, neurodegenerative disorders, including those associated with aging, and prodromal depression associated with dementia.

The present chimeric nitrate esters also demonstrate an additional, and important benefit, i.e., a substantial reduction in the therapeutic lag period associated with antidepressant drugs. Brain-derived neurotrophic factor (BDNF) is a gene product of CREB that regulates neurogenesis and neuronal plasticity, mediates neuroprotection, and is associated with memory formation (P. Ernfors et al., Trends Neurosci 2003, 26, 171-173.; S. W. Ying et al., J Neurosci 2002, 22, 1532-1540.; and M. Gooney et al., J Neurochem 2001, 77, 1198-1207). NO increases BDNF production, and BDNF induces neuronal NO synthase (nNOS) expression (A. Cheng et al., Dev Biol 2003, 258, 319-333). Control of BDNF levels is intrinsic to the control of depression, and delayed elevation of BDNF has been associated with the therapeutic lag time in the onset of action of antidepressant drugs. A present chimeric nitrate ester elevates BDNF levels rapidly after initiation of treatment, and reduces the therapeutic lag period from about 2-6 weeks to about 2-7 days.

SSRI antidepressants, like fluoxetine, have a well-described clinical history and are prescribed for a range of neuropsychiatric disorders in addition to general depression. Current theories place delayed upregulation of BDNF as a cause of the 2-6 week delay in onset of SSRI antidepressant action, and the BDNF mediated increase in plasticity and neurogenesis as a central mechanism of action of SSRIs. NO mimetic nitrates reverse cognition deficits in animal models and activate the ERK/CREB cascade. In addition, it is theorized, but not relied upon that NO mimetics also regulate BDNF activity. Therefore, a chimeric nitrate ester containing an SSRI pharmacophore maintains or enhances antidepressant activity and aids cognition. A present chimeric nitrate ester provides ancillary pathways for enhancement of cognition and neuronal and synaptic plasticity, and provides a therapeutic benefit in the treatment of dementia and depression.

The present invention therefore provides chimeric nitrate esters, i.e., organic nitrates that contain an antidepressant pharmacophore and an NO mimetic. A present chimeric nitrate ester incorporating an antidepressant pharmacophore (e.g., an SSRI) represents a novel antidepressant therapy. For example, initial elevation of serotonin levels and long-term elevation of BDNF leading to neurogenesis are linked to the therapeutic efficacy of SSRI drugs. In one embodiment, the present invention provides chimeric nitrate esters having SSRI activity, which provide precognitive and antidepressant activity. The present nitrate esters exhibit benefits beyond those of traditional SSRIs and classic nitrate drugs.

The present invention therefore provides compounds, compositions, and methods useful in the treatment of depression and diseases of aging where depression and dementia are comorbid. The present methods comprise administering a therapeutically effective amount of a chimeric nitrate ester of an antidepressant drug, such as a compound of a structural formula (I), (II), or (III), including salts, prodrugs, hydrates, or solvates thereof, to a subject in need thereof.

In one embodiment, a present chimeric nitrate has a structural formula (I):

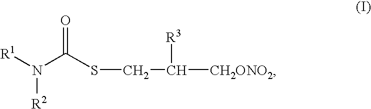

wherein R¹ is selected from the group consisting of C₁₋₆alkyl, C₃₋₈cycloalkyl, C₃₋₈heterocycloalkyl, aryl, and heteroaryl;

R² is selected from the group consisting of C₁₋₆alkyl, C₃₋₈cycloalkyl, C₃₋₈heterocycloalkyl, aryl, and heteroaryl, and

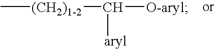

R¹ and R² are taken with the nitrogen atom to which they are attached to form a 5- or 6-membered ring, said ring substituted with an aryl or heteroaryl group and optionally with —(CH₂)_{1,2}—O-aryl; and

R³ is H or ONO₂,

or a pharmaceutically acceptable salt, prodrug, hydrate, or solvate thereof.

In another embodiment, a present chimeric nitrate ester has a structural formula (II):

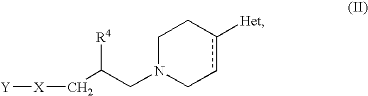

wherein X is null, O, or S;

R⁴ is —OH or —ONO₂;

Y is —CH₂aryl; —(CH₂)₁₋₂ONO₂, or

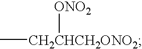

Het is heteroaryl; and

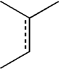

is a carbon-carbon single bond or a carbon-carbon double bond,

or a pharmaceutically acceptable salt, prodrug, hydrate, or solvate thereof.

In yet another aspect, a present nitrate ester has a structural formula (III):

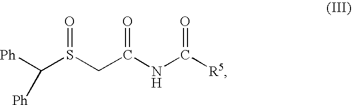

wherein Ph is phenyl; and

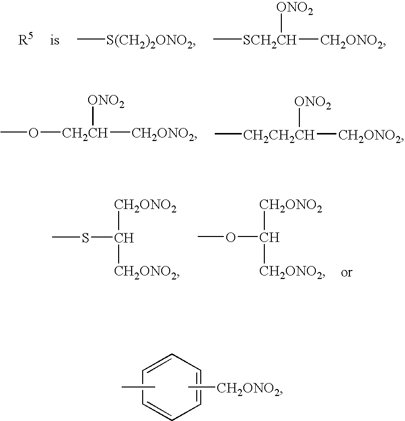

or a pharmaceutically acceptable salt, prodrug, hydrate, or solvate thereof.

In one preferred embodiment, a present chimeric nitrate ester has a nitrate moiety positioned β, γ, or δ to an organosulfur functionality.

The compounds of structural formula (I), (II), and (III) exhibit antidepressant activity, are NO mimetics, and are useful in a treatment of depression. The method comprises administering a therapeutically effective amount of a chimeric nitrate ester having an antidepressant pharmacophone, such as compound of structural formula (I), (II), or (III), to an individual in need thereof.

As used herein, the term “alkyl” refers to straight chained and branched saturated hydrocarbon groups, nonlimiting examples of which include methyl, ethyl, and straight chain and branched propyl, butyl, pentyl, and hexyl groups. The term “alkylene” refers to an alkyl group having a substituent. The term (CH₂)₁₋₂ refers to CH₂ or CH₂CH₂.

As used herein, groups such as

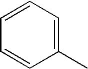

is an abbreviation for

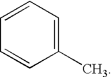

As used herein, groups such as C₁₋₃alkylphenyl means a C₁₋₃alkyl group bonded to a phenyl ring, for example,

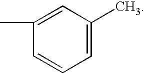

Groups such as C₁₋₃alkylenephenyl means a phenyl group bonded to a C₁₋₃alkylene group, for example

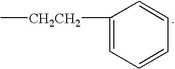

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four, groups independently selected from, for example, halo, alkyl, alkoxy, —CF₃, —OCF₃, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkoxy, —CF₃, —OCF₃, aryl, and heteroaryl. Examples of heteroaryl groups include, but are not limited to, thienyl, furyl, oxazolyl, quinolyl, thiophenyl, isoquinolyl, indolyl, triazinyl, triazolyl, isothiazolyl, isoxazolyl, imidazolyl, benzothiazolyl, pyrimidinyl, thiazolyl, thiadiazolyl, pyridinyl, pyridazinyl, pyrazolyl, pyrazinyl, quinolyl, tetrazolyl, oxazolyl, pyrrolyl, benzimidazolyl, benzofuranyl, cinnolinyl, indazolyl, indolizinyl, phthalazinyl, triazinyl, isoindolyl, purinyl, oxadiazolyl, furazanyl, benzofurazanyl, benzothiophenyl, benzotriazolyl, benzothiazolyl, benzisothiazolyl, benzoxazolyl, benzisoxazolyl, benzimidazolyl, quinazolinyl, quinoxalinyl, napththyridinyl, dihydroquinolyl, tetrahydroquinolyl, dihydroisoquinolyl, tetrahydroisoquinolyl, benzofuryl, furopyridinyl, pyrrolopyrimidinyl, and azaindolyl.

As used herein, the term “C₃₋₈cycloalkyl” means a monocyclic aliphatic ring containing three to eight carbon atoms.

As used herein, the term “heterocycloalkyl” means a monocyclic or a bicyclic aliphatic ring containing 5 to 10 total atoms, of which one to five of the atoms are independently selected from nitrogen, oxygen, and sulfur and the remaining atoms are carbon.

In accordance with the present invention, preferred compounds of structural and formula (I) are those wherein:

R¹ is C₁₋₆ alkyl, and preferably C₁₋₃ alkyl;

p is 0 or 1;

R² is

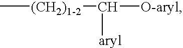

wherein the moiety —Oaryl optionally is substituted with a CF₃ group;

R³ is H or ONO₂; or

R¹ and R² are taken together with the nitrogen atom to which they are attached to form a 6-membered ring, wherein the 6-membered ring contains one substituent selected from (a) aryl optionally substituted with a halo and (b) heteroaryl, and further contains an optional —CH—O-aryl group, wherein said aryl group is selected from

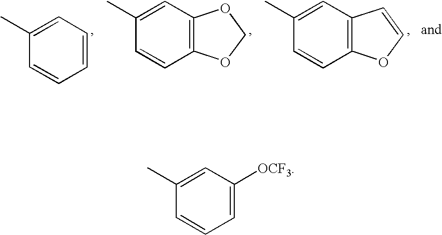

In further accordance with the present invention, preferred compounds of structural formula (II) are those wherein:

X is null, O, or S;

Y is CH₂aryl,

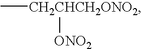

### or —CH₂ONO₂;

R⁴ is —OH or —ONO₂;

Het is

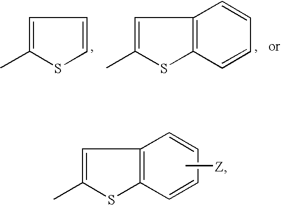

wherein Z is selected from the group consisting of halo, alkyl, alkoxy, CF₃, and OCF₃; and

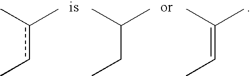

Additionally, salts, prodrugs, hydrates, and solvates of the present chimeric nitrate esters also are included in the present invention and can be used in the methods disclosed herein. The present invention further includes all possible stereoisomers and geometric isomers of the chimeric nitrate esters, such as the compounds of structural formula (I), (II), and (III). The present invention includes both racemic compounds and optically active isomers. When a chimeric nitrate ester is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the chimeric nitrate ester are possible, the present invention is intended to include all tautomeric forms of the compounds.

Prodrugs of the chimeric nitrate esters, such as compounds of structural formula (I), (II), and (III), are included in the present invention. It is well established that a prodrug approach, wherein a compound is derivatized into a form suitable for formulation and/or administration, then released as a drug in vivo, has been successfully employed to transiently (e.g., bioreversibly) alter the physicochemical properties of the compound (see, H. Bundgaard, Ed., “Design of Prodrugs,” Elsevier, Amsterdam, (1985); R. B. Silverman, “The Organic Chemistry of Drug Design and Drug Action,” Academic Press, San Diego, chapter 8, (1992); K. M. Hillgren et al., Med. Res. Rev., 15, 83 (1995)).

Compounds of the present invention can contain one or more functional groups. The functional groups, if desired or necessary, can be modified to provide a prodrug. Suitable prodrugs include, for example, acid derivatives, such as amides and esters. It also is appreciated by those skilled in the art that N-oxides can be used as a prodrug.

Compounds of the present invention can exist as salts. Pharmaceutically acceptable salts of the compounds of the invention often are preferred in the methods of the invention. As used herein, the term “pharmaceutically acceptable salts” refers to salts or zwitterionic forms of a present chimeric nitrate ester, such as the compounds of structural formula (I), (II), or (III). Salts of the present compounds can be prepared during the final isolation and purification of the compounds or separately by reacting the compound with an acid having a suitable cation. The pharmaceutically acceptable salts of a present chimeric nitrate ester can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the invention include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphosphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present invention appearing herein is intended to include a chimeric nitrate ester, such as compounds of structural formula (I), (II), or (III), as well as pharmaceutically acceptable salts, hydrates, solvates, or prodrugs thereof.

The chimeric nitrates of the present invention also can be conjugated or linked to auxiliary moieties that promote a beneficial property of the compound in a method of therapeutic use. Such conjugates can enhance delivery of the compounds to a particular anatomical site or region of interest (e.g., a tumor), enable sustained therapeutic concentrations of the compounds in target cells, alter pharmacokinetic and pharmacodynamic properties of the compounds, and/or improve the therapeutic index or safety profile of the compounds. Suitable auxiliary moieties include, for example, amino acids, oligopeptides, or polypeptides, e.g., antibodies, such as monoclonal antibodies and other engineered antibodies; and natural or synthetic ligands to receptors in target cells or tissues. Other suitable auxiliaries include fatty acid or lipid moieties that promote biodistribution and/or uptake of the compound by target cells (see, e.g., Bradley et al., Clin. Cancer Res. (2001) 7:3229).

The following are examples of chimeric nitrate esters of the present invention:

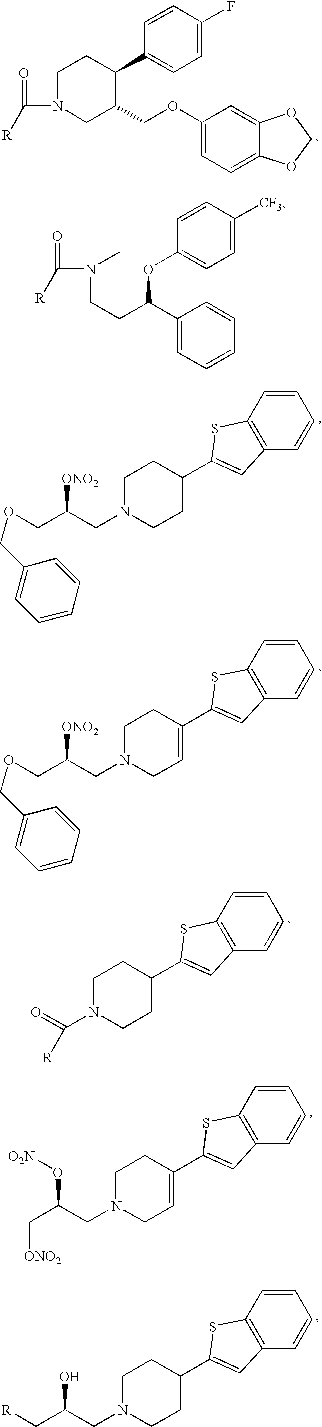

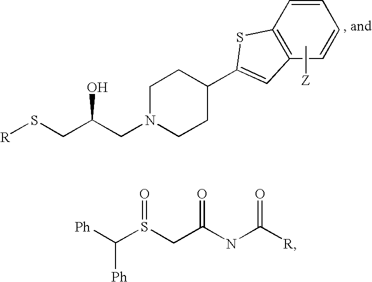

wherein R is selected from the group consisting of

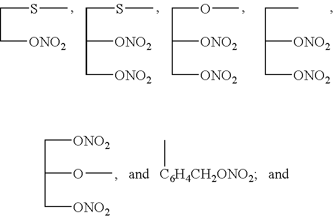

Z is selected from the group consisting of halo, alkyl, alkoxy, CF₃, and OCF₃.

Specific compounds of the present invention include, but are not limited to,

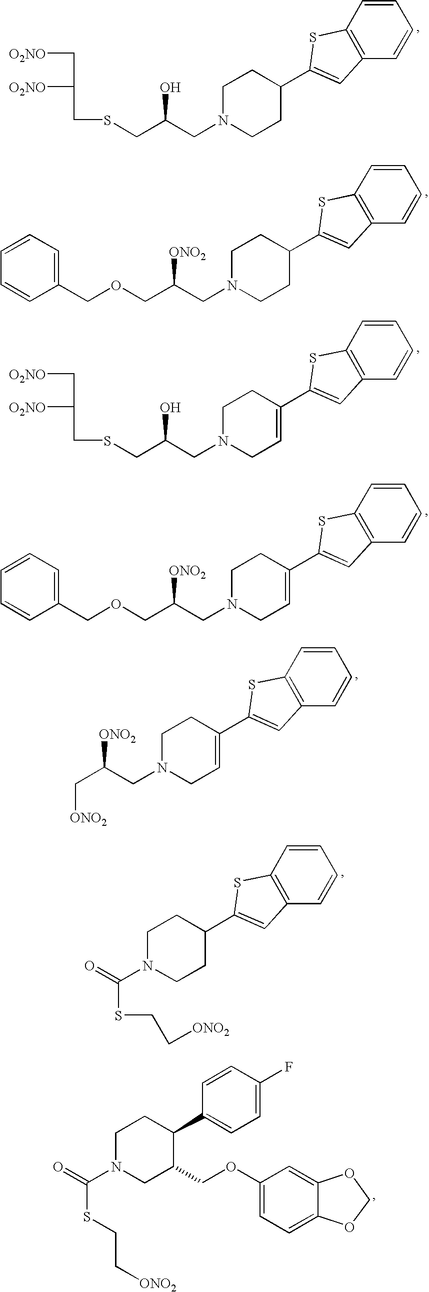

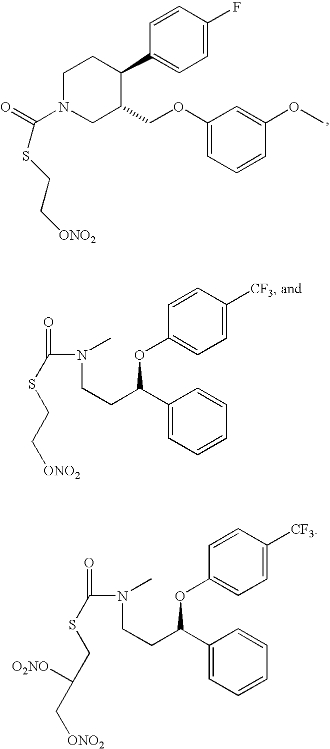

In one embodiment, the present invention provides a method of treating depression by administering a therapeutically effective amount of chimeric nitrate ester of the present invention to an individual in need thereof. The method of the present invention can be accomplished by administering a present chimeric nitrate ester as the neat compound or as a pharmaceutical composition. Administration of a pharmaceutical composition, or neat chimeric nitrate ester, can be performed during or after the onset of depression. Typically, the pharmaceutical compositions are sterile, and contain no toxic, carcinogenic, or mutagenic compounds that would cause an adverse reaction when administered.

The present invention therefore is directed to compounds and methods of treating depression. The present invention also is directed to pharmaceutical compositions comprising a present chimeric nitrate ester useful in the treatment of depression. Further provided are kits comprising a present chimeric nitrate ester, and an insert having instructions for using the chimeric nitrate ester.

Within the meaning of the present invention, the term “depression” encompasses a wide variety in the type and severity of the condition. The spectrum for depression varies from a condition that is transient and temporary, e.g., lasting a few days, to a clinical, general depression that can be chronic. Depression can be characterized by persistent severe feelings of worthlessness, guilt, sadness, helplessness, and hopelessness. Common symptoms can include inactivity, difficulty thinking or concentrating, appetite changes, sleep disturbances, and suicidal tendencies. Cognitive deficits, including impaired memory and disrupted attention, are frequently reported in patients suffering from depression.

Depression also is a symptom of a large variety of disorders, including all dementias, and is especially prevalent in, for example, Parkinson's disease, epilepsy, and cancer. Depression also accompanies life changes not associated with a pathophysiology, such as peri- and postmenopause and postnatum.

Signs and symptoms of depression treatable by the present methods are not only negative thoughts, moods, and behaviors, but also are specific changes in bodily functions, for example, crying spells, body aches, low energy, low libido, as well as problems with eating, weight, or sleeping.

Major depression is treatable by the present methods and is characterized by a one or more symptom that lasts for at least two weeks, including a sad and/or irritable mood, or that interfere with an ability to work, sleep, eat, and enjoy once-pleasurable activities.

Dysthymia is a less severe, but usually more long-lasting, type of depression that also can be treated in accordance with the present invention. This condition involves chronic symptoms that do not disable, but prevent, the affected person from functioning effectively or from feeling well. Individuals suffering from dysthymia also can experience episodes of major depression, which is referred to as “double-depression”.

The present invention therefore provides compounds and methods to alleviate the signs and symptoms of depression, including, but not limited to, a persistently sad, anxious, angry, irritable, and/or “empty” mood; feelings of hopelessness and/or pessimism; feelings of guilt, worthlessness, and/or helplessness; loss of interest or pleasure in hobbies and activities that once were enjoyed, including sex; insomnia, early-morning awakening, and/or oversleeping; decreased appetite and/or weight loss, or overeating and/or weight gain; increased fatigue, decreased energy, and/or being “slowed down”; crying spells; thoughts of death and/or suicide and/or suicide attempts; restlessness and/or irritability; difficulty in concentrating, remembering, and/or making decisions; and persistent physical symptoms that do not respond to treatment, such as headache, digestive disorders, and/or chronic pain.

Children and adolescents with depression also are treatable and may experience the symptoms described above, but further may exhibit other symptoms in lieu of, or in addition to, the symptoms listed above, including: poor school performance; persistent boredom; and/or frequent complaints of physical problems, such as headaches and stomach aches. Some of the classic “adult” symptoms of depression also may be more easily diagnosed in young individuals, such as a change in eating or sleeping patterns. Teen depression further can be characterized by the adolescent taking more risks and/or showing less concern for their own safety.

In the present method, a therapeutically effective amount of one or more chimeric nitrate ester of the invention, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A chimeric nitrate ester of the invention can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a chimeric nitrate ester of the invention is administered in an effective amount to achieve its intended purpose. The exact formulation, route of administration, and dosage is determined by an individual physician in view of the diagnosed depression. Dosage amount and interval can be adjusted individually to provide levels of a chimeric nitrate ester of the invention that is sufficient to maintain therapeutic effects.

Toxicity and therapeutic efficacy of a chimeric nitrate ester of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. Compounds that exhibit high therapeutic indices are preferred. The data obtained from such data can be used in formulating a dosage range for use in humans. The dosage preferably lies within a range of circulating compound concentrations that include the ED₅₀ with little or no toxicity. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a present chimeric nitrate ester required for use in therapy varies with the nature of the depression being treated, the length of time that activity is desired, and the age and the condition of the patient, and ultimately is determined by the attendant physician. Dosage amounts and intervals can be adjusted individually to provide plasma levels of the nitrate ester that are sufficient to maintain the desired therapeutic effects. The desired dose conveniently can be administered in a single dose, or as multiple doses administered at appropriate intervals, for example as one, two, three, four or more subdoses per day. For example, a present chimeric nitrate ester can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

The dosage of a composition containing a present chimeric nitrate ester, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 g/kg. The dosage of a composition may be at any dosage including, but not limited to, about 1 μg/kg, 10 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, or 200 mg/kg. The above dosages are exemplary of the average case, but there can be individual instances in which higher or lower dosages are merited, and such are within the scope of this invention. In practice, the physician determines the actual dosing regimen that is most suitable for an individual patient, which can vary with the age, weight, and response of the particular patient.

A chimeric nitrate ester used in a method of the present invention can be administered in an amount of about 0.005 to about 200 milligrams per dose, about 0.05 to about 150 milligrams per dose, or about 0.5 to about 100 milligrams per dose. For example, a present chimeric nitrate ester, can be administered, per dose, in an amount of about 0.005, 0.05, 0.5, 5, 10, 20, 25, 30, 40, 50, 60, 70, 80, 100, 150, or 200 milligrams, including all doses between 0.005 and 200 milligrams.

The chimeric nitrate esters typically are administered in admixture with a pharmaceutical carrier selected with regard to the intended route of administration and standard pharmaceutical practice. Pharmaceutical compositions for use in accordance with the present invention are formulated in a conventional manner using one or more physiologically acceptable carriers comprising excipients and auxiliaries that facilitate processing of chimeric nitrate esters.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a present chimeric nitrate ester is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a present chimeric nitrate ester. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a present chimeric nitrate ester.

When a therapeutically effective amount of a present chimeric nitrate ester is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle. A present chimeric nitrate ester can be infused with other fluids over a 10-30 minute span or over several hours.

A present chimeric nitrate ester can be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the chimeric nitrate ester to a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A present chimeric nitrate ester can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water soluble form. Additionally, suspensions of a present chimeric nitrate ester can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

A present chimeric nitrate ester also can be formulated in rectal compositions, such as suppositories or retention enemas, e.g., containing conventional suppository bases. In addition to the formulations described previously, a present chimeric nitrate ester also can be formulated as a depot preparation. Such long-acting formulations can be administered by implantation (for example, subcutaneously or intramuscularly) or by intramuscular injection. Thus, for example, a present chimeric nitrate ester can be formulated with suitable polymeric or hydrophobic materials (for example, as an emulsion in an acceptable oil) or ion exchange resins.

In particular, a present chimeric nitrate ester can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The chimeric nitrate esters also can be injected parenterally, for example, intravenously, intramuscularly, subcutaneously, or intracoronarily. For parenteral administration, the chimeric nitrate esters are best used in the form of a sterile aqueous solution which can contain other substances, for example, salts or monosaccharides, such as mannitol or glucose, to make the solution isotonic with blood.

As an additional embodiment, the present invention includes kits which comprise one or more chimeric nitrate ester or composition containing the same packaged in a manner that facilitates their use to practice methods of the invention. In one embodiment, the kit includes a chimeric nitrate ester or composition containing the same as useful for practice of a method, packaged in a container, such as a sealed bottle or vessel, with a label affixed to the container or included in the kit that describes use of the chimeric nitrate ester or composition to practice the method of the invention. Preferably, the chimeric nitrate ester or composition is packaged in a unit dosage form. The kit further can include a device suitable for administering the composition according to the intended route of administration.

### Synthesis of Chimeric Nitrate Esters

Compounds of the present invention were prepared as follows. In particular, the following illustrates the synthesis of chimeric nitrate esters that contain, for example, an SSRI or modafinil moiety. Control compounds also were prepared that do not contain an antidepressant pharmacophone.

SSRIs were selected as the antidepressant pharmacophore in these nonlimiting examples because of their preferred use in the treatment of chronic depression, safety profile, and evidence for regulation of BDNF and neurogenesis. In particular, paroxetine and fluoxetine were selected for incorporation into a present chimeric nitrate ester. A benzothiophene based pharmacophore, developed as a dual SSRI-5HT_{1A} receptor antagonist, also was incorporated into a present nitrate ester of an antidepressant.

In addition, a modafinil-based chimeric nitrate ester was prepared. Modafinil is a non-addictive, clinical anti-narcolepsy drug used widely off-label as an antidepressant. Modafinil has shown promise as an alternative to stimulant augmentation of antidepressant therapy in patients with comorbid conditions. Another antidepressant pharmacophore is based on tianeptine.

In one embodiment, mononitrate compounds of the present invention were prepared as shown in the following scheme, which avoids S-oxidation:

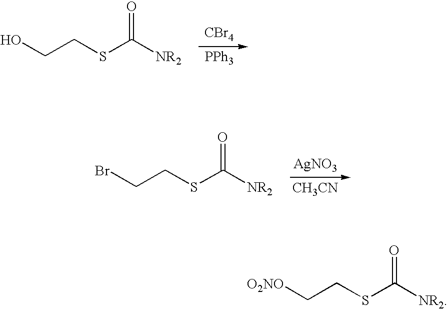

In accordance with the present invention, the resulting thiocarbamate is a labile linker liberating the antidepressant pharmacophore R₂NH and the organic nitrate. The organic nitrate provides NO mimetic activity, as illustrated below for a present chimeric nitrate ester having fluoxetine as the antidepressant pharmacophore, i.e., NO-fluoxetine.

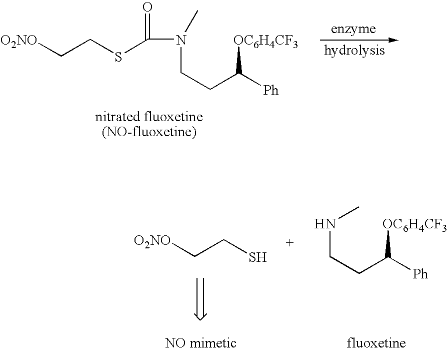

Unless otherwise stated all temperatures are in degrees Celsius. Also, in these examples and elsewhere, abbreviations have the following meanings:

| | |
|---|---|
| tBuLi | tertiary butyl lithium |
| Boc | tert-butoxycarbonyl |
| NaHCO₃ | sodium bicarbonate |
| CH₂Cl₂ | dichloromethane |
| CDCl₃ | deuterated chloroform |
| m | multiplet |
| s | singlet |
| MHz | megahertz |
| δ | chemical shift |
| J | coupling constant in Hertz |
| dd | doublet of doublets |
| d | doublet |
| t | triplet |
| Li | lithium |
| CaH₂ | calcium hydride |
| THF | tetrahydrofuran |
| Ar | argon |
| N₂ | nitrogen |
| ppm | parts per million |
| Me₄Si | tetramethylsilane |
| h | hour |
| min | minute |
| rt | room temperature |
| NH₄Cl | ammonium chloride |
| Na₂SO₄ | sodium sulfate |
| mmol | millimole |
| mM | millimolar |
| μM | micromolar |
| mL | milliliter |
| μL | microliter |
| g | gram |
| mg | milligram |
| NaH | sodium hydride |
| H₂ | hydrogen gas |
| HNO₃ | nitric acid |
| Ac₂O | acetic anhydride |
| KSCN | potassium thiocyanate |
| DTT | dithiothreitol |
| Ph₃P | triphenylphosphate |
| MeOH | methanol |
| kg | kilogram |
| NaCl | sodium chloride |
| KCl | potassium chloride |
| KH₂PO₄ | potassium dihydrogen phosphate |
| CaCl₂ | calcium chloride |
| MgSO₄ | magnesium sulfate |
| HEPES | 4-(2-hydroxyethyl)-1-piperazineethane sulfonic acid |
| SDS | sodium dodecyl sulfate |
| cm | centimeter |
| DMSO | dimethyl sulfoxide |
| GSH | glutathione |

Chemicals were purchased from Aldrich or Acros Chemical Corporation and used without further purification. Solvents were distilled before use: CH₂Cl₂ was distilled from CaH₂, and THF was distilled from sodium wire. All reactions were performed under inert atmosphere (Ar or N₂) unless otherwise noted. NMR spectra were recorded with a Varian Unity INOVA 300 at 300 MHz (¹H), 75 MHz (¹³C) at 25° C. ¹H chemical shifts (δ) were reported in ppm with Me₄Si (δ=0.00 ppm) or CDCl₃ (δ=7.26 ppm) as internal standards. ¹³C chemical shifts were reported with CDCl₃ (δ=77.00 ppm) or Me₄Si (δ=0.00 ppm) as internal standards. Thin layer chromatography (TLC) was performed on Merck silica gel 60 F₂₅₄ glass plates. Column chromatography was performed using Merck silica gel (35-75 mesh) and ethyl acetate/hexanes as eluent.

### Preparation of Compound 1

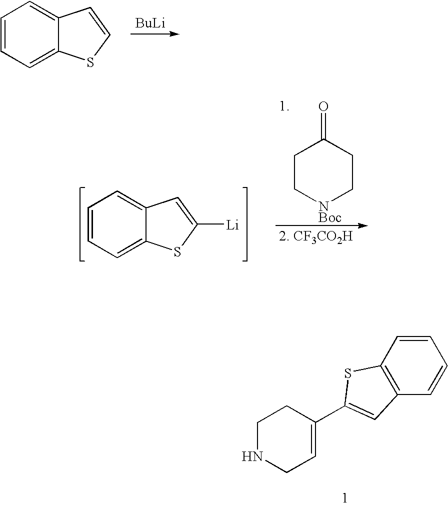

A solution of benzo[b]thiophene (3.35 g, 25 mmol) in anhydrous THF (100 mL) was cooled to −78° C. (acetone-dry ice bath), then t-BuLi (10 mL, 2.5 M in THF) was added dropwise within 30 min. After the addition was complete, the yellow suspension was allowed to stir at the same temperature for another 30 min. A solution of tert-butyl-4-oxopiperidine-1-carboxylate (4.0 g, 20 mmol) in anhydrous THF (100 mL) was added slowly via syringe to the above suspension while maintaining the inner temperature below −65° C. The mixture was allowed to warm and stir at rt overnight. The next morning, the mixture was cooled again to 0° C. and saturated aqueous NH₄Cl (20 mL) was added. Most of the solvent was removed under reduced pressure, and the residue was extracted with ethyl acetate (100 mL×3). The organic layers were combined, washed with brine (100 mL×3), and dried over Na₂SO₄. After filtration and concentration, the resulting yellow syrup (7.05 g) was dissolved in methylene chloride (150 mL) and cooled to 0° C. before trifluoroacetic acid (15 mL) was added dropwise. The brown solution was allowed to stir at rt overnight, then transferred into a 250 mL separatory funnel. The organic layer was washed successively with water (50 mL×3), saturated aqueous NaHCO₃ (50 mL×3), and brine (50 mL×3). After drying over Na₂SO₄, the organic layer was concentrated and further purified by flash chromatography to provide compound 1 (4.63 g, 86%) as a brown syrup, which solidifies slowly at rt.

¹H NMR (300 MHz, CDCl₃) δ 7.73 (d, J=6.7 Hz, 1H), 7.67 (d, J=6.7 Hz, 1H), 7.27 (dd, J=3.0 Hz, J₂=1.6 Hz, 2H), 7.13 (s, 1H), 6.25 (s, 1H), 3.53 (d, J=2.4 Hz, 2H), 3.10 (t, J=5.6 Hz, 2H), 2.52 (br, 2H);

¹³C NMR (75 MHz, CDCl₃) δ 145.54, 140.22, 138.25, 130.28, 125.37, 124.27, 124.20, 123.26, 121.98, 118.10, 45.24, 42.87, 27.44.

### Preparation of Compound 3

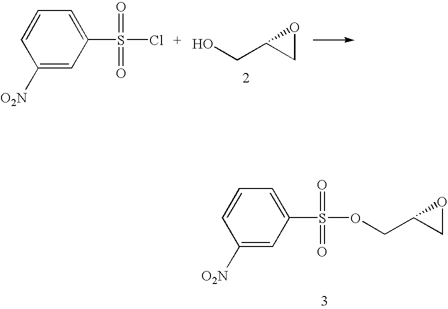

To a cooled (0° C., ice bath) solution of compound 2 (220 mg, 3.0 mmol) in anhydrous methylene chloride (10 mL) and triethylamine (1.5 mL, 10 mmol) was added 3-nitrobenzene-1-sulfonyl chloride (730 mg, 3.3 mmol) in small portions. Compound 2 is commercially available. The brown solution was allowed to stir at 0° C. for 1 h, then warmed to rt. The reaction was monitored by TLC every 15 min before all compound 2 was completely consumed. Crushed ice (1-2 g) was added to quench the reaction. The reaction mixture was transferred to a separatory funnel and partitioned between methylene chloride (20 mL) and water (10 mL). The organic layer was washed successively with saturated aqueous NaHCO₃ (10 mL×3) and brine (10 mL×3), then dried over Na₂SO₄. After filtration and concentration, the residue was further purified by flash column chromatography to afford compound 3 as a brown solid (710 mg, 92%).

¹H NMR (400 MHz, CDCl₃) δ 8.77 (s, 1H), 8.54 (d, J=8.1 Hz, 1H), 8.28 (d, J=7.9 Hz, 1H), 4.50 (d, J=11.5 Hz, 1H), 4.04 (dd, J=4.9 Hz, J₂=1.5 Hz, 1H), 3.23 (d, J=1.8 Hz, 1H), 2.85 (dd, J₁=4.0 Hz, J₂=2.0 Hz, 1H), 2.64 (dd, J₁=2.4 Hz, J₂=2.0 Hz, 1H);

¹³C NMR (100 MHz, CDCl₃) δ 148.19, 137.92, 133.32, 130.81, 128.39, 123.16, 71.69, 48.64, 44.42.

### Preparation of Compound 4

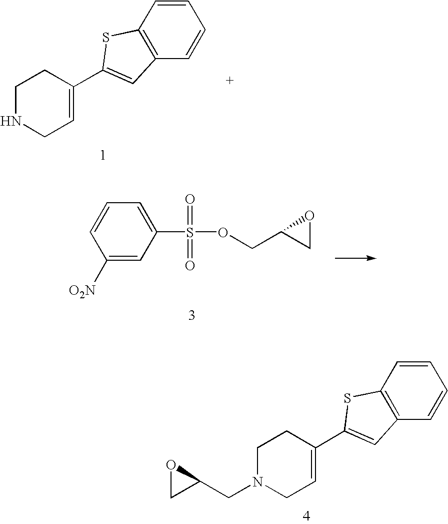

Compound 1 (220 mg, 1.0 mmol) was dissolved in anhydrous THF (10 mL). The solution was cooled to 0° C. and NaH (40 mg, 1.0 mmol, 60% in mineral oil) was added in one portion. The gray suspension was stirred at the same temperature for about 30 min until H₂ ceased bubbling. To the above suspension at 0° C. was added dropwise a solution of compound 3 (280 mg, 1.1 mmol) in anhydrous THF (6 mL). The mixture was allowed to warm to rt and stirred overnight. The next morning, crushed ice (1-2 g) was added to quench the reaction, and the mixture was extracted with ethyl acetate (20 mL×3). The organic layers were combined and washed successively with saturated aqueous NH₄Cl (20 mL×3), brine (20 mL×3) and dried over Na₂SO₄. After filtration and concentration under reduced pressure, the crude syrup was purified by flash column chromatography to give compound 4 (170 mg, 63%) as a brown solid.

¹H NMR (300 MHz, CDCl₃) δ 7.74 (d, J=8.0 Hz, 1H), 7.64 (d, J=6.5 Hz, 1H), 7.28 (dd, J=6.5 Hz, J₂=4.0 Hz, 2H), 7.14 (s, 1H), 6.21 (s, 1H), 3.41 (dd, J₁=9.4 Hz, J₂=6.2 Hz, 2H), 3.16-3.18 (m, 1H), 2.88 (dd, J₁=8.3 Hz, J₂=5.4 Hz, 2H), 2.80 (td, J=5.4 Hz, J₂=4.4 Hz, 2H), 2.66-2.68 (m, 2H), 2.53 (t, J=2.4 Hz, 2H);

¹³C NMR (75 MHz, CDCl₃) δ 147.76, 144.21, 137.27, 131.08, 124.77, 124.20, 123.93, 123.26, 121.06, 119.67, 47.47, 46.45, 44.58, 42.65, 36.10, 27.34.

### Preparation of Compound 5

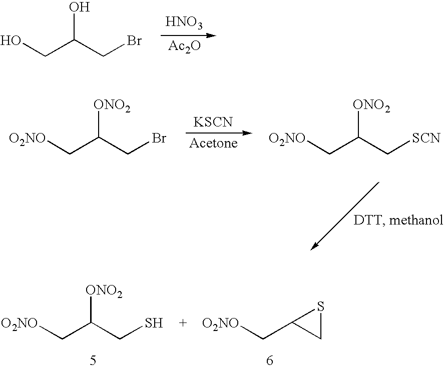

For experimental details of the preparation of compound 5, see: Thatcher et al. Org. Lett; 2001, 3(8), 1113-8.

¹H NMR (300 MHz, CDCl₃) δ 5.30-5.32 (m, 1H), 4.89 (AB, J₁=5.3 Hz, J₂=3.3 Hz, 1H), 4.72 (A′B′, J₁=5.3 Hz, J₂=3.3 Hz, 1H), 2.83-2.93 (m, 2H).

### Preparation of a Chimeric Nitrate Ester

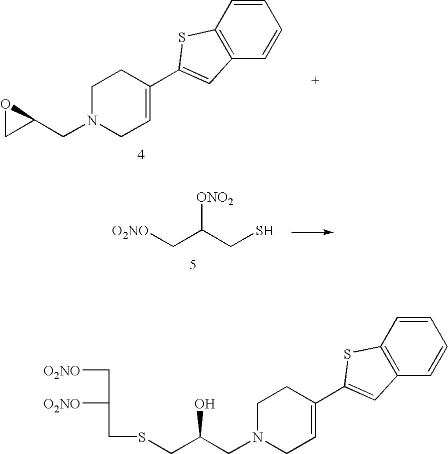

To a solution of compound 4 (150 mg, 0.55 mmol) in methanol (5.0 mL) was added compound 5 (550 mg, 2.76 mmol). The mixture was stirred vigorously at rt overnight. The next morning, TLC showed some unconsumed starting material 4 remained. The mixture was refluxed at 70° C. (oil bath) for 2 h. The solvent was removed under reduced pressure, and the residue was purified by flash column chromatography to afford the chimeric nitrate ester (137 mg, 53%) as a yellow-brown solid. Compound 4 also was recovered (46 mg, 31%). Compound 6 was obtained as a by-product (difficult to measure the amount because of its volatility).

¹H NMR (300 MHz, CDCl₃) δ 7.76 (d, J=8.1 Hz, 1H), 7.69 (d, J=6.4 Hz, 1H), 7.30 (dd, J₁=7.6 Hz, J₂=3.8 Hz, 2H), 7.18 (s, 1H), 6.17 (s, 1H), 4.50-4.52 (m, 2H), 4.09-4.11 (m, 2H), 3.68 (t, J=5.6 Hz, 1H), 2.67-2.70 (m, 4H), 1.78-1.79 (m, 2H), 1.64-1.66 (m, 2H);

¹³C NMR (75 MHz, CDCl₃) δ144.76, 144.21, 140.20, 138.04, 129.99, 128.31, 127.66, 124.77, 124.30, 123.93, 73.42, 72.53, 66.51, 60.29, 52.91, 49.99, 44.04, 42.71, 35.10, 27.62.

The extraction of paroxetine from commercially available paroxetine tablets.

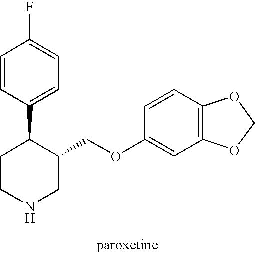

Two hundred paroxetine tablets (each containing 20 mg of paroxetine) were added to a 500 mL round-bottom flask, and saturated NaHCO₃ (aq, 200 mL) was added. The mixture was stirred at rt vigorously overnight. The next morning, the slurry suspension was transferred to a 500 mL separatory funnel and extracted with methylene chloride (150 mL×3). The organic layers were combined and dried over Na₂SO₄. After filtration and concentration under reduced pressure, the syrup was further purified by flash column chromatography to provide paroxetine as a colorless syrup (1.76 g) which solidifies slowly at rt.

¹H NMR (300 MHz, CDCl₃) δ 7.21 (m, 2H), 6.99 (m, 2H), 6.61 (d, J=8.0 Hz, 1H), 6.33 (d, J=2.4 Hz, 1H), 6.13 (dd, J=8.2 Hz, J=2.5 Hz, 1H), 5.88 (s, 2H), 3.75 (dd, J₁=13.0 Hz, J₂=3.6 Hz, 1H), 3.67 (m, 1H), 3.61 (dd, J₁=9.7 Hz, J₂=3.7 Hz, 1H), 3.48 (dd, J₁=9.7 Hz, J₂=3.8 Hz, 1H), 3.20 (t, J=12.0 Hz, 1H), 3.04 (m, 1H), 2.90 (m, 1H), 2.68 (m, 1H), 2.41 (m, 1H), 2.02 (m, 1H).

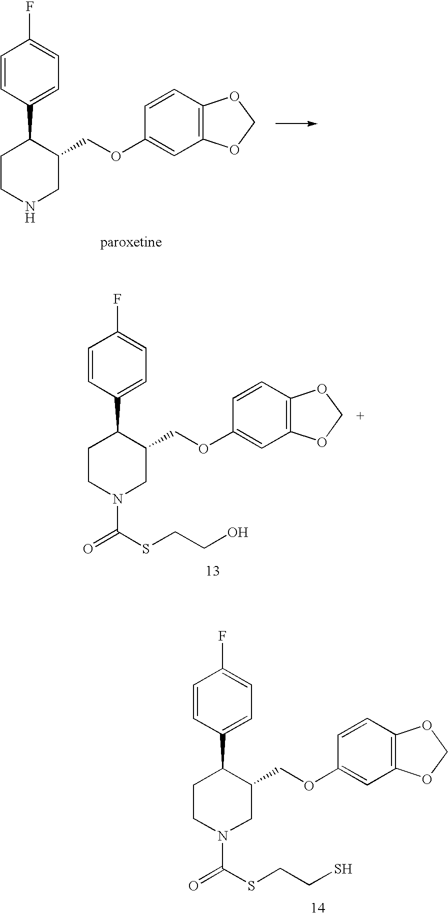

### Method 1:

To a solution of paroxetine (560 mg, 1.70 mmol) in anhydrous methylene chloride (10 mL) and triethylamine (1.40 mL, 10 mmol) at 0° C. was added phosgene (0.85 mL, 20% in toluene). The mixture was stirred at 0° C. for 2 h and warmed to rt. After stirring at rt for 10 h, the mixture again was cooled to 0° C., and a solution of 2-mercaptoethanol (0.15 mL, 2.0 mmol) in methylene chloride (0.5 mL) was added dropwise. The reaction mixture was allowed to warm to rt slowly and stirred at rt overnight. The next morning, the mixture was transferred into a 125 mL separatory funnel and partitioned between methylene chloride (30 mL) and water (15 mL). The organic layer was washed successively with water (15 mL×3) and brine (15 mL×3). The organic layer was dried over Na₂SO₄ and filtrated. The filtrate was concentrated under reduced pressure to give a residue that was further purified by column chromatography to afford compound 13 (440 mg, 60%) and compound 14 (292 mg, 38%) as syrups.

### Compound 13

¹H NMR (300 MHz, CDCl₃) δ 7.08 (m, 2H), 6.90 (m, 2H), 6.55 (d, J=7.8 Hz, 1H), 6.29 (d, J=2.4 Hz, 1H), 6.07 (dd, J=8.0 Hz, J=2.5 Hz, 1H), 5.80 (s, 2H), 3.78 (t, J=4.6 Hz, 2H), 3.61 (dd, J₁=9.7 Hz, J=3.7 Hz, 1H), 3.54 (m, 1H), 3.48 (dd, J₁=9.7 Hz, J₂=3.8 Hz, 1H), 3.39 (t, J=12.0 Hz, 1H), 3.04 (m, 1H), 2.90 (m, 1H), 2.84 (m, 1H), 2.71 (m, 1H), 2.68 (m, 1H), 2.41 (m, 1H), 2.02 (m, 1H);

¹³C NMR (75 MHz, CDCl₃) δ 164.37, 162.06, 161.38, 154.76, 148.88, 142.03, 138.53, 138.27, 128.85, 128.70, 115.78, 115.64, 108.26, 107.44, 102.34, 99.04, 68.68, 44.97, 44.20, 34.67, 33.87, 32.05.

### Compound 14

¹H NMR (300 MHz, CDCl₃) δ 7.09 (m, 2H), 6.91 (m, 2H), 6.53 (d, J=8.2 Hz, 1H), 6.26 (d, J=2.2 Hz, 1H), 6.05 (dd, J=8.0 Hz, J=2.1 Hz, 1H), 5.78 (s, 2H), 4.03 (t, J=7.2 Hz, 2H), 3.80 (d, J=12.2 Hz, 1H), 3.53 (t, J=7.0 Hz, 2H), 3.40 (d, J=7.0 Hz, 1H), 2.83 (m, 2H), 2.57 (t, J=5.7 Hz, 2H), 2.1 (m, 1H), 1.72 (m, 2H).

### Method 2

To a solution of paroxetine (500 mg, 1.52 mmol) in anhydrous methylene chloride (10 mL) and triethylamine (0.4 mL, 3.1 mmol) was added 1,1′-carbonyldiimidazole (265 mg, 1.52 mmol) in small portions. The suspension was stirred under an argon atmosphere at rt overnight and concentrated. The residue was dissolved in anhydrous THF (10 mL) and cooled to 0° C. With vigorous stirring, methyl iodide (0.1 mL, 1.7 mmol) was added dropwise and the resulting mixture was allowed to stir at rt overnight. The next morning, the solvent was removed under reduced pressure and the crude product was dissolved in anhydrous acetonitrile (10 mL) and cooled to 0° C. A solution of 2-mercaptoethanol (0.15 mL, 2.0 mmol) in anhydrous acetonitrile (2 mL) was added dropwise, and the mixture was stirred at rt for 24 h. After removal of the solvent under reduced pressure, ethyl acetate (30 mL) was added to dilute the crude product, and the organic layer was washed with water (10 mL×3) and brine (10 mL×3) successively. The organic layer was dried over Na₂SO₄ and filtered and concentrated. The residue was purified by column chromatography to afford compound 13 (598 mg, 91%) and compound 14 (30 mg, 4.7%) as syrups.

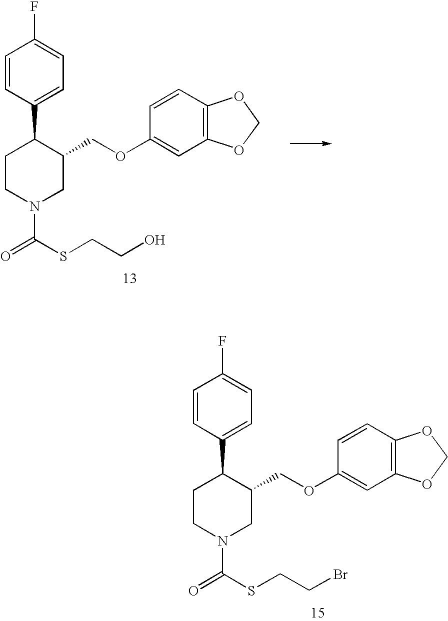

To a solution of compound 13 (310 mg, 0.71 mmol) in anhydrous chloroform (10 mL) at 0° C. was added Ph₃P (223 mg, 0.85 mmol). The solution was stirred at the same temperature for 15 min before carbon tetrabromide (352 mg, 1.06 mmol) was added in small portions. The mixture was stirred at 0° C. for 2 h and slowly warmed up to rt. The clear yellow solution was allowed to stir at rt overnight, then diluted with chloroform (20 mL) and transferred to a 125 mL separatory funnel. The organic layer was washed with brine (10 mL×3) and dried over Na₂SO₄. After filtration and concentration, the yellow residue was further purified by flash column chromatography to afford compound 15 (345 mg, 98%) as a yellow solid.

¹H NMR (300 MHz, CDCl₃) δ 7.10 (dd, J₁=5.7 Hz, J₂=2.8 Hz, 2H), 6.96 (dd, J₁=8.1 Hz, J₂=6.4 Hz, 2H), 6.61 (dd, J₁=8.4 Hz, J₂=2.3 Hz, 1H), 6.34 (d, J=2.3 Hz, 1H), 6.12 (d, J=8.4 Hz, 1H), 5.86 (s, 2H), 3.59 (d, J=9.4 Hz, 1H), 3.53 (t, J=7.3 Hz, 2H), 3.46 (dd, J₃=6.8 Hz, J₂=2.1 Hz, 1H), 3.35 (t, J=6.5 Hz, 2H), 3.04 (m, 1H), 2.72 (m, 1H), 2.02 (d, J=2.4 Hz, 2H), 1.88 (m, 2H), 1.72 (m, 1H);

¹³C NMR (75 MHz, CDCl₃) δ 165.46, 162.91, 160.47, 153.99, 148.18, 141.82, 138.29, 138.26, 128.75, 128.68, 115.74, 115.53, 107.84, 105.57, 101.13, 97.99, 68.39, 43.95, 42.06, 33.63, 32.41, 31.05.

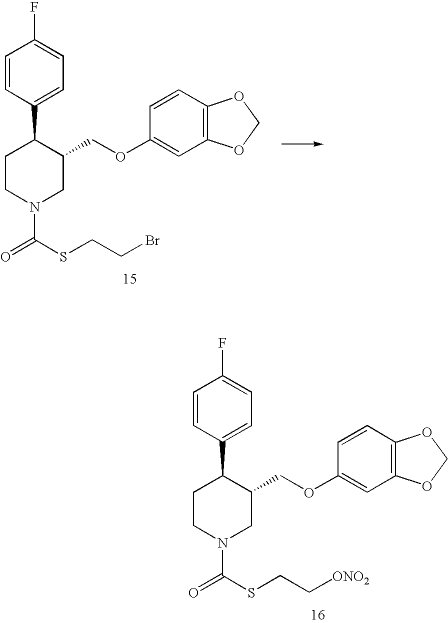

To a solution of compound 15 (150 mg, 0.30 mmol) in anhydrous acetonitrile (5 mL) was added silver nitrate (85 mg, 0.5 mmol). The flask and condenser were covered with aluminum foil, and the reaction mixture was refluxed at 80° C. (oil bath) for 6 h, then cooled to rt. The dark brown precipitate was filtered off through a short silica gel column (1 g silica gel). The filtrate was concentrated and condensed under reduced pressure. The resulting brown residue was further purified by flash column chromatography to afford compound 16 (138 mg, 97%) as a syrup, which slowly solidifies at rt.

¹H NMR (300 MHz, CDCl₃) δ 7.06 (d, J=5.0 Hz, 2H), 6.89 (dd, J₁=8.0 Hz, 2=2.2 Hz, 2H), 6.54 (d, J=8.2 Hz, 1H), 6.28 (d, J=2.1 Hz, 1H), 6.06 (d, J=7.4 Hz, 1H), 5.78 (s, 2H), 4.55 (t, J=4.9 Hz, 2H), 3.53 (t, J=8.4 Hz, 1H), 3.38 (dd, J₃=8.7 Hz, J₂=6.0 Hz, 1H), 3.16 (t, J=5.8 Hz, 2H), 3.02 (m, 1H), 2.7 (m, 2H), 1.99 (m, 2H), 1.79 (m, 1H), 1.66 (m, 1H);

¹³C NMR (75 MHz, CDCl₃) δ 165.12, 162.78, 160.34, 153.88, 148.09, 141.72, 138.20, 128.68, 128.60, 115.61, 115.40, 107.73, 105.48, 101.03, 97.89, 71.61, 68.29, 43.80, 41.92, 33.51, 27.03.

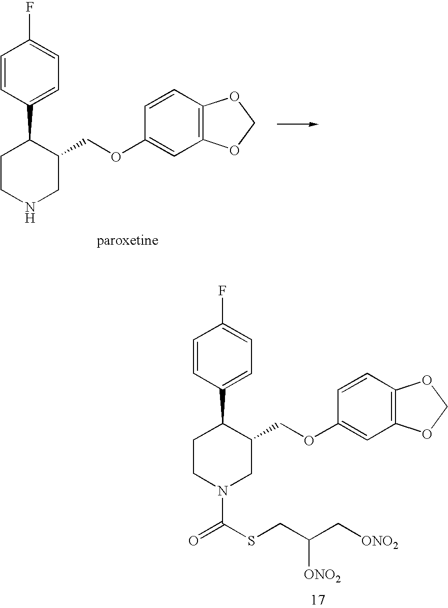

To a solution of paroxetine (200 mg, 0.61 mmol) in anhydrous methylene chloride (5 mL) and triethylamine (0.5 mL, 3.6 mmol) at 0° C. was added phosgene (0.36 mL, 0.70 mmol, 20% in toluene). The mixture was stirred at 0° C. for 2 h and warmed to rt. After stirring at rt for 10 h, the mixture was cooled to 0° C. and a solution of compound 5 (240 mg, 1.2 mmol) in methylene chloride (0.5 mL) was added dropwise. The resulting mixture was allowed to warm to rt slowly and stirred at rt overnight. The next morning, the mixture was transferred into a 125 mL separatory funnel and partitioned between methylene chloride (30 mL) and water (15 mL). The organic layer was washed successively with water (15 mL×3) and brine (15 mL×3). The organic layer was dried over Na₂SO₄ and filtered. The filtrate was concentrated under reduced pressure to give a residue which was further purified by column chromatography to afford 17 (219 mg, 65%) as a syrup. Starting material paroxetine (35 mg) also was recovered.

¹H NMR (300 MHz, CDCl₃) δ 7.14 (dd, J₁=8.1 Hz, J₂=2.8 Hz, 2H), 6.97 (dd, J₁=7.8 Hz, J₂=6.4 Hz, 2H), 6.68 (d, J=8.4 Hz, 1H), 6.31 (d, J=2.1 Hz, 1H), 6.05 (d, J=8.4 Hz, 1H), 5.86 (s, 2H), 3.51 (d, J=8.7 Hz, 1H), 3.55 (t, J=6.8 Hz, 2H), 3.47 (dd, J₁=7.8 Hz, J₂=2.1 Hz, 1H), 3.35 (t, J=6.5 Hz, 2H), 3.04 (m, 1H), 2.72 (m, 1H), 2.02 (d, J=2.4 Hz, 2H), 1.88 (m, 2H), 1.72 (m, 1H);

¹³C NMR (75 MHz, CDCl₃) δ 167.46, 163.45, 161.58, 154.41, 148.71, 141.65, 137.73, 135.46, 127.65, 124.31, 115.70, 109.43, 108.80, 104.37, 104.01, 101.25, 99.64, 97.52, 69.08, 43.55, 42.06, 34.40, 31.14.

The extraction of fluoxetine from commercially available fluoxetine tablets.

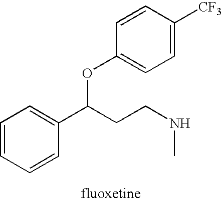

The procedure to extract fluoxetine is described above for the extraction of paroxetine.

¹H NMR (300 MHz, CDCl₃) δ 7.32 (d, J=7.9 Hz, 2H), 7.22-7.24 (m, 4H), 7.15 (d, J=7.8 Hz, 1H), 6.81 (d, J=7.6 Hz, 2H), 5.17 (m, 1H), 2.64 (m, 2H), 2.32 (s, 3H), 1.91 (m, 2H);

¹³C NMR (75 MHz, CDCl₃) δ 160.52, 141.01, 128.69, 128.62, 128.38, 127.73, 115.70, 81.36, 78.57, 78.21, 78.14, 53.31, 51.43, 48.15, 40.58, 38.67, 36.62.

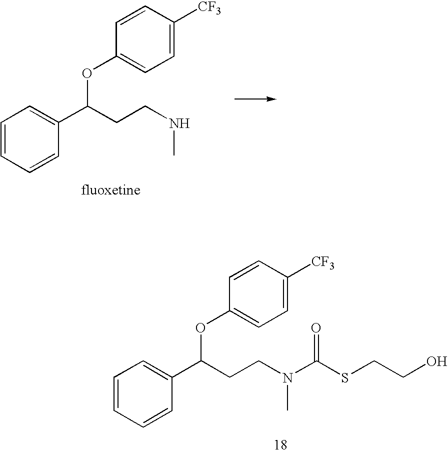

The procedure to prepare compound 18 is similar to the procedure for preparing compound 13.

¹H NMR (300 MHz, CDCl₃) δ 7.35 (d, J=8.2 Hz, 2H), 7.20-7.24 (m, 5H), 6.81 (d, J=8.6 Hz, 2H), 5.13 (t, J=5.0 Hz, 1H), 3.61 (t, J=7.4 Hz, 2H), 3.53 (t, J=6.8 Hz, 2H), 3.02 (s, 3H), 2.94 (m, 2H), 2.17 (t, J=5.7 Hz, 2H);

¹³C NMR (75 MHz, CDCl₃) δ 160.01, 149.66, 149.65, 140.02, 139.84, 128.98, 128.91, 128.21, 126.81, 125.58, 115.67, 49.70, 48.58, 38.87, 36.97, 36.84, 35.97.

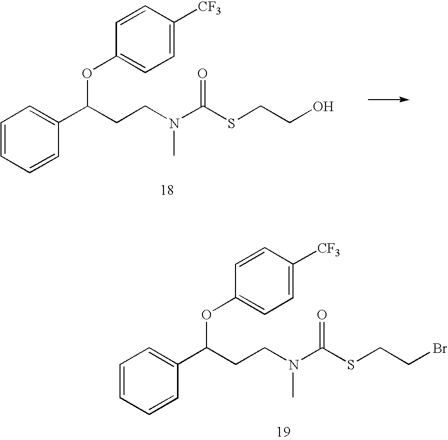

The procedure to prepare compound 19 is similar to the procedure for preparing compound 15.

¹H NMR (300 MHz, CDCl₃) δ 7.35 (d, J=8.1 Hz, 2H), 7.20-7.23 (m, 4H), 7.19 (d, J=4.6 Hz, 1H), 6.81 (d, J=8.2 Hz, 2H), 5.11 (t, J=5.2 Hz, 1H), 3.52 (t, J=6.9 Hz, 2H), 3.49 (m, 2H), 3.18 (t, J=7.0 Hz, 2H), 2.91 (s, 3H), 2.15 (t, J=6.8 Hz, 2H);

¹³C NMR (75 MHz, CDCl₃) δ 160.13, 128.90, 128.06, 127.08, 126.80, 126.76, 126.73, 126.69, 125.55, 122.92, 115.68, 115.43, 46.82, 36.35, 35.13, 32.37, 30.82.

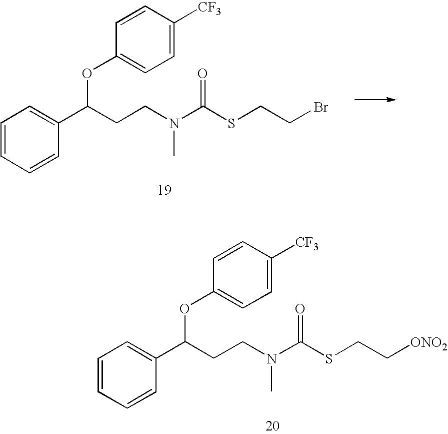

The procedure to prepare compound 20 is similar to the procedure for preparing compound 16.

¹H NMR (300 MHz, CDCl₃) δ 7.36 (d, J=8.5 Hz, 2H), 7.18-7.22 (m, 5H), 6.81 (d, J=8.5 Hz, 2H), 5.11 (t, J=3.8 Hz, 1H), 4.42 (m, 2H), 3.52 (t, J=6.8 Hz, 2H), 3.06 (t, J=6.3 Hz, 2H), 2.92 (s, 3H), 2.13 (t, J=7.8 Hz, 2H);

¹³C NMR (75 MHz, CDCl₃) δ 160.13, 140.35, 128.92, 128.09, 126.78, 126.73, 125.56, 122.47, 122.41, 115.68, 78.03, 77.68, 71.51, 46.88, 36.38, 35.12, 27.11.

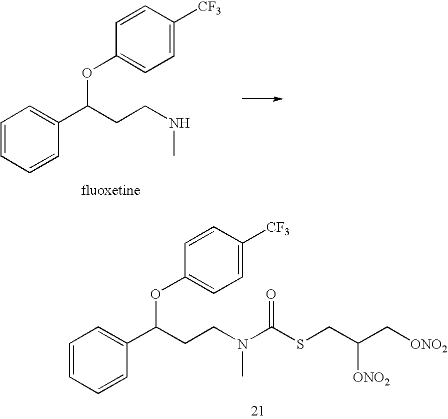

The procedure to prepare 21 is similar to the procedure for preparing compound 17.

¹H NMR (300 MHz, CDCl₃) δ 7.36 (d, J=8.0 Hz, 2H), 7.19-7.22 (m, 5H), 6.80 (d, J=7.8 Hz, 2H), 5.10 (t, J=4.7 Hz, 1H), 4.58 (t, J=2.7 Hz), 4.40 (m, 2H), 3.54 (t, J=6.5 Hz, 2H), 3.06 (t, J=6.5 Hz, 2H), 2.92 (s, 3H), 2.13 (t, J=7.8 Hz, 2H);

¹³C NMR (75 MHz, CDCl₃) δ 160.42, 141.75, 128.81, 128.09, 126.55, 125.73, 125.04, 123.28, 123.05, 122.41, 118.47, 79.06, 77.85, 71.64, 46.57, 46.03, 37.26, 35.11, 27.28.

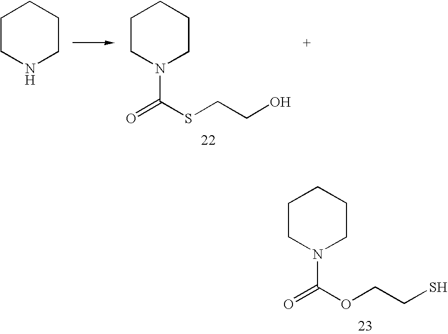

To a solution of piperidine (0.20 mL, 2.0 mmol) in anhydrous methylene chloride (5 mL) and triethylamine (1.40 mL, 10 mmol) at 0° C. was added phosgene (1.05 mL, 20% in toluene, 2.0 mmol). The mixture was stirred at 0° C. for 2 h and warmed to rt. After stirring rt for 10 h, the mixture was cooled to 0° C. and a solution of 2-mercaptoethanol (0.15 mL, 2.0 mmol) in methylene chloride (0.5 mL) was added dropwise. The reaction system was allowed to warm to rt slowly and stirred at rt overnight. The next morning, the mixture was transferred into a 125 mL separatory funnel and partitioned between methylene chloride (20 mL) and water (10 mL). The organic layer was washed successively with water (10 mL×3) and brine (10 mL×3). The organic layer was dried over Na₂SO₄ and filtered. The filtrate was concentrated under reduced pressure to give a residue, which was further purified by column chromatography, to afford compound 22 (288 mg, 76%) and compound 23 as colorless oils.

### Compound 22

¹H NMR (300 MHz, CDCl₃) δ 3.98 (t, J=8.4 Hz, 2H), 3.94 (t, J=8.2 Hz, 2H), 3.17 (t, J=2.4 Hz, 4H), 2.81 (tt, J₁=7.3 Hz, J₂=4.6 Hz, 4H), 2.36 (m, 2H).

¹³C NMR (75 MHz, CDCl₃) δ 61.91, 60.38, 52.26, 44.24, 37.18, 34.52.

### EXAMPLES

The following nonlimiting examples and experiments illustrate the new and unexpected benefits provided by the chimeric nitrate esters of the present invention. In addition to efficacy in treating depression, another unexpected benefit is a substantial decrease in the therapeutic lag period associated with current antidepressant drugs.

In particular, current therapies for depression are characterized by a delay in clinical onset of antidepressant activity. This delay parallels a delay in BDNF production, which is believed to play a central role in antidepressant activity. The NO/sGC/cGMP/ERK pathway is implicated in CREB phosphorylation and BDNF production. The present chimeric nitrate esters activate this pathway in rat hippocampus with acute drug administration, which is an effective approach to shorten the delay of BDNF production and to speed the process of clinical antidepressant activity.

The present chimeric nitrate esters are antidepressant drugs with a quick onset of antidepressant action. For example, the bioactivation of a nitrated fluoxetine, i.e., NO-fluoxetine, was determined using LC-MS in rat liver microsome and plasma. The activation kinetics of NO-fluoxetine also was determined in buffers. NO-fluoxetine was active in a modified Porsolt forced swim test and an in vitro assay for selective serotonin reuptake inhibition. NO-mimetic activity was investigated in an in vivo model of depression.

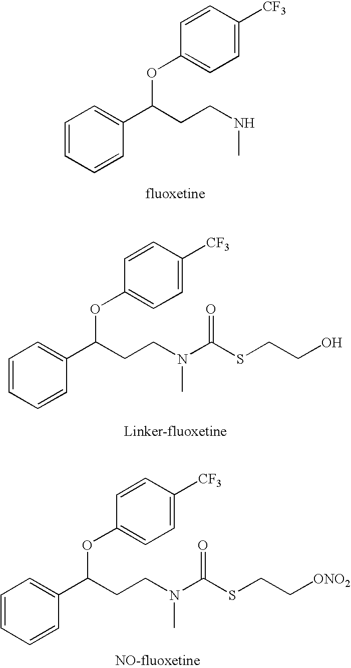

### NO-Fluoxetine Bioactivation

In Phosphate buffer saline A stock solution of NO-fluoxetine was prepared by dissolving 1 mg of the compound in 100 μL MeOH (21 mM). An aliquot (10 μL) of the stock solution was added to 990 μL of 50 mM phosphate buffer (pH 7.4) at 22° C., and the formation of fluoxetine was monitored by reverse phase HPLC using 4.6 mm×150 mm C18 column at a flow rate of 1.0 mL/min. The mobile phase contained 60% acetonitrile content, which was increased to 90% over 5 min. After the phase was held 90% for 5 min, the acetonitrile was reduced to 60% in 5 min. Formation of fluoxetine in buffers was positively identified by comparing retention time and the LC-MS (electrospary ionization) protonated [M+H]+ molecular ion (m/z 310) of the authentic sample. Fluoxetine formation was monitored every 15 min and percentage of fluoxetine formation was obtained with respect to time. Kinetic experiments were performed in 50 mM phosphate buffer solutions (pH 7.0, pH 7.4, and pH 8.0).

In rat liver microsomes A solution containing substrate (0.25 mM), rat liver microsomes (3 nmol of P450/0.5 mL), and NADPH (2 mM) in 50 mM phosphate buffer (pH 7.4, 0.5 mL total volume) was incubated for 30 min at 37° C. The reaction was quenched by addition of acetonitrile and centrifugation for 10 min. The solution was filtered and injected to HPLC.

In plasma A solution containing substrate (0.25 mM) and rat plasma (0.1 mL) was incubated for 30 min at 37° C. The reaction was quenched by addition of acetonitrile and centrifugation for 10 min. The solution was filtered and injected to HPLC.

NOx detection A degassed solution containing NO-fluoxetine (0.5 mM), and cysteine (5 mM) or GSH (5 mM) in 50 mM phosphate buffer (pH 7.4, 1 mL total volume) was incubated for 30 min at 37° C. After 30 min, nitric oxide was detected using an NO analyzer, and nitrite and nitrate was detected using Griess assay.

Serotonin reuptake inhibition assay Human embryonal kidney (HEK-293) cell line stably transfected with serotonin transporter (SERT) cDNA were used to determine the serotonin reuptake inhibitory activity. Cells were cultured in Dulbecco's Modified Eagle's Medium (Gibco BRL., Grand Island, N.Y.) containing 10% dialyzed fetal bovine serum (Gibco BRL, Grand Island, N.Y.), 1% Glutamax, 1% penicillin-streptomycin, and 600 μg/ml Geneticin. Cells were plated at a concentration of 15×10⁴ cells/well in 24-well plates coated with poly-D-Lysine and allowed to grow at 37° C. for 24 hours. Growth medium was aspirated and each well was rinsed with Krebs-Ringer-Henseleit (KRH) buffer (5.6 mM glucose, 125 mM NaCl, 4.8 mM KCl, 1.2 mM KH₂PO₄, 1.2 mM CaCl₂, 1.2 mM MgSO₄, 25 mM HEPES, pH 7.4). Cells then were incubated in 0.45 ml KRH buffer for 10 min at 37° C. in the presence of SSRIs. Cells were re-incubated for 10 minutes after the addition of 50 ul ³[H]5-HT mixture (200 nM ³[H]5-HT corresponding to 2.46 Ci/mmol, 0.1 mM ascorbic acid, and 0.1 mM pargyline HCl in KRH buffer). Uptake was terminated by aspirating the reaction mixture and washing the cells three times with KRH buffer. Cells then were solubilized with 1% SDS, and radioactivity was quantified by liquid scintillation counting using Beckman LS 5801 scintillation counter.

Modified forced swimming test This test was performed using male Sprague Dawley rats weighing 200-250 g. Rats were trained to swim in the water tank for 15 minutes 24 hour before the test. NO-Fluoxetine was administered by i.p. injections 23.5, 5, and 1 hour before the test at a dose of 26.5 mg/kg, which is equimolar to 20 mg/kg fluoxetine. NO-fluoxetine was freshly prepared in an emulsion formed of 5% Tween 80. The test was performed by putting the rats in a water tank consisting of an acrylic cylinder (22 cm diameter, 30 cm water depth, 25° C. water temperature) for 5 minutes. The 5 minute test was divided into 60 blocks of 5 seconds each. The major behavior in each 5 sec block was assigned for the whole block, including climbing, swimming, and immobile. The blocks representing different behaviors were counted.

Step-through passive avoidance (STPA) task This test was performed on male C57BL/6 mice (from Charles River's Laboratories), 8-10 weeks of age and weighing 22-27 g. The mice were kept under standardized laboratory conditions (temperature 21±1° C., humidity 50-60%) with free access to food and tap water in a room with a natural light-dark cycle.

The passive avoidance task is regarded in the art as a measure involving long-term memory. Avoidance experiment was divided into 3 phases: habituation, training, and retention. The apparatus used consisted of a box divided into 2 compartments, an illuminated compartment adjacent to a dark compartment, the floor consisted of an electric grid controlled by a switch that can deliver an electric shock only to the dark compartment. A doorway was located at floor level in the center of the connecting wall; the door was closed once the mouse enters the dark compartment. The habituation phase was performed by individually putting the mice in the light compartment; the door was closed once the mouse enters the dark compartment (without an electric shock), left there for 15 seconds before being returned to its cage. The time (latency) to enter the dark box was recorded for each mouse; only mice scoring less than 30 seconds for three consecutive times were allowed to pass for the training phase. Training phase was performed 2 hours after the habituation phase by individually placing the mice again in the illuminated compartment but an electric shock (0.6 mA for 2 s) was delivered immediately after the mouse entered the dark compartment accompanied by the door closure. Trial was repeated for a maximum of 5 trials or until the mouse remained in the light compartment for 300 seconds in one of the trials. During the training phase, the number of trials and the time (latency) to enter the dark compartment was scored. Mice that entered the dark compartment in the fifth trial in less than 300 seconds were eliminated from the experiment. Forty-eight hours after the training trial, a retention test was conducted in which the same animals were put into the illuminated box and again the latency to enter the dark box was recorded. The retention trial ended when the mouse entered the dark box or 300 s had elapsed, whichever occurred first. Scopolamine (1 mg/kg) dissolved in physiological saline water was administered by i.p. injection 30 minutes before the training phase and drugs dissolved in 25% DMSO were administered by i.p. injection 20 minutes before the training.

Bioactivation of NO-fluoxetine NO-Fluoxetine was converted to fluoxetine in 50 mM phosphate buffer solutions (pH 7.0, 7.4, and 8.0). Formation of fluoxetine in buffer was positively identified by comparison to the retention time and the LC-MS (electrospary ionization) protonated [M+H]+ molecular ion (m/z 310) of an authentic fluoxetine sample.

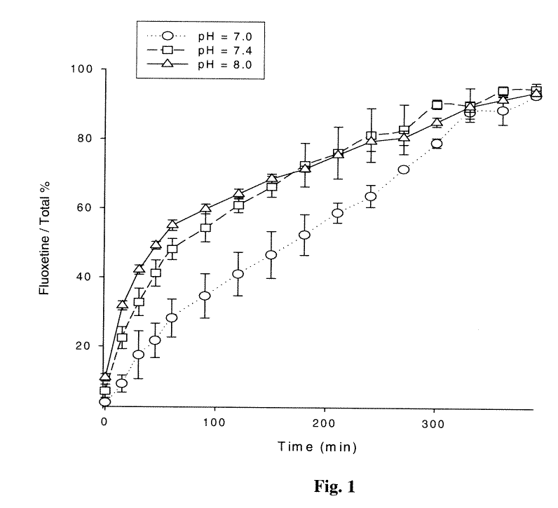

Kinetic studies for the degradation of NO-fluoxetine were performed by monitoring the production of fluoxetine every 15 min in different pH buffer solutions (See FIG. 1). The half life of fluoxetine in pH 7.0, 7.4, and 8.0 buffer solutions was 190 min, 90 min, and 45 min, respectively. The reaction of NO-fluoxetine with rat liver microsomes and plasma also generated fluoxetine as the major product.

The bioactivation of NO-fluoxetine in a phosphate buffer is illustrated below, together with a proposed, but not relied upon, mechanism of bioactivation.

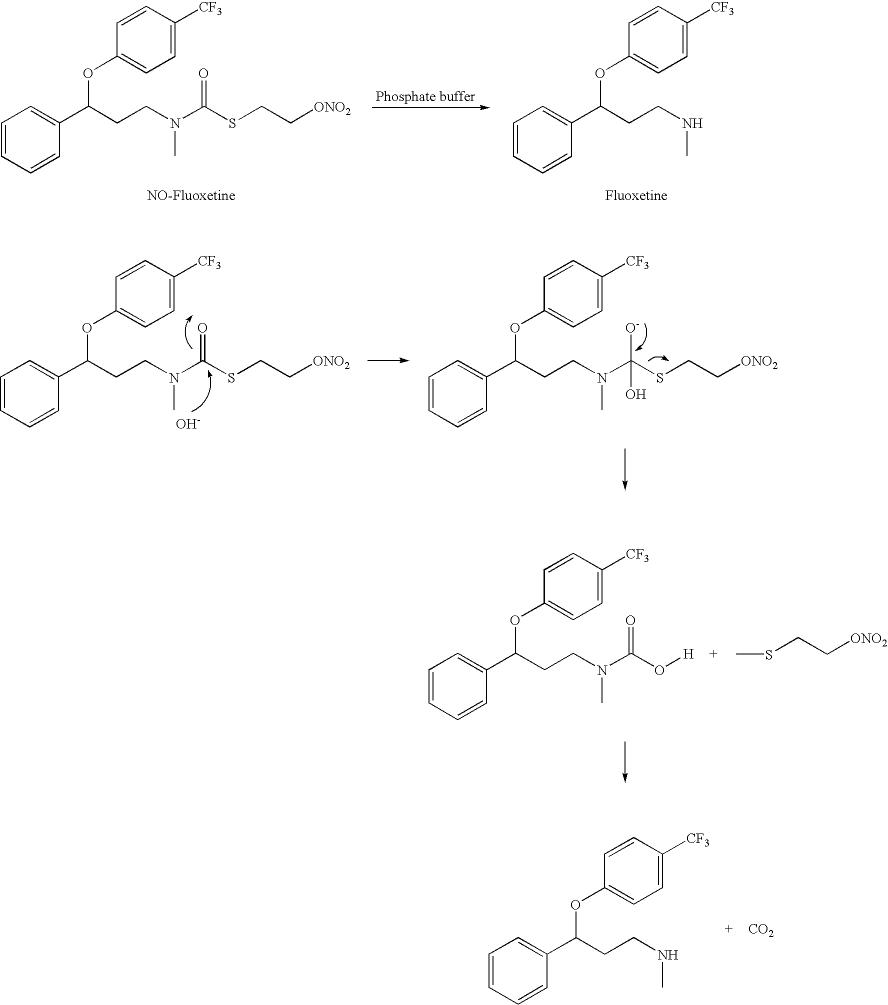

### Antidepressant Activity of NO-Fluoxetine

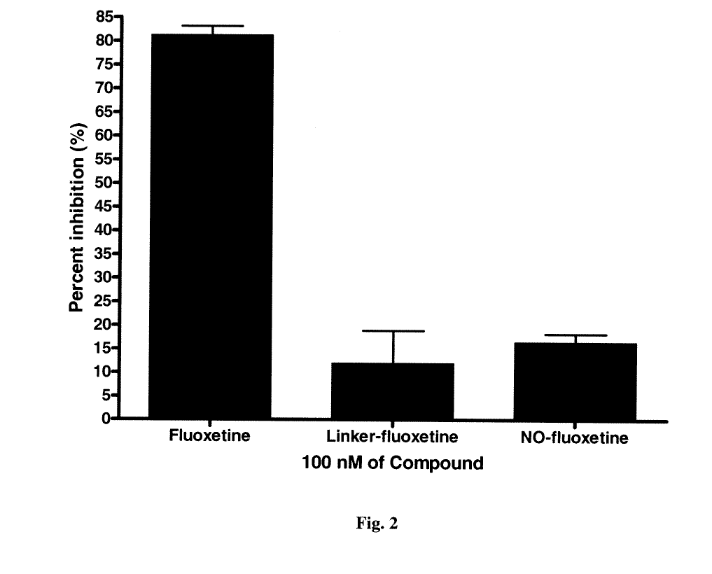

In vitro NO-fluoxetine and linker-fluoxetine showed a reduced activity in the serotonin reuptake inhibition assay (FIG. 2), 16.55+/−1.74% and 12.04+/−6.83% respectively, compared to fluoxetine, 81.29+/−1.84% at 100 nM. In particular, FIG. 2 shows the in vitro serotonin reuptake inhibitory activity of fluoxetine, linker-fluoxetine, and NO-fluoxetine. Activity was determined by comparing H³-5HT transported into HEK cells stably transfected with human SERT and incubated with the drug for 10 minutes prior to H³-5HT exposure. 100% inhibition was obtained using 10 uM of fluoxetine. The extent of H³-5HT transported into the cell was determined by measuring the radioactivity of the cell lysates using a liquid scintillation counter. This indicates that the IC₅₀ of NO-fluoxetine and linker-fluoxetine are higher than 100 nM (IC₅₀ of fluoxetine is 9.08 nM). Incubation time prior to serotonin addition is relatively short (10 minutes) compared to the half life of fluoxetine formation (90 minutes at pH 7.4) which makes the data obtained representing the activity of the pro-drug rather than fluoxetine.

In vivo NO-fluoxetine (26.5 mg/kg equimolar to 20 mg/kg fluoxetine) given by i.p. injections 23.5, 5, and 1 hour before the test was active in the modified forced swimming test, showing an increase in the swimming behaviour and a decrease in the immobility behaviour. Sub-acute injection of NO-fluoxetine provides sufficient time for the bioactivation of NO-fluoxetine and the subsequent release of fluoxetine.

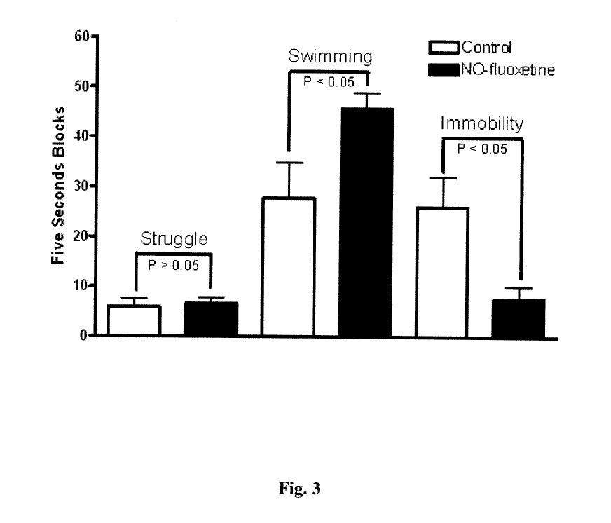

FIG. 3 shows the effect of NO-fluoxetine on the duration of struggle, swimming, and immobility in the modified forced swimming test. Values presented as mean±SEM, n=6 per group. The data were statistically evaluated by ANOVA, followed by Turkey post-hoc test.

### Memory Restoring Effect of NO-fluoxetine

2.05 mg/kg of NO-fluoxetine (equimolar to 1 mg/kg) given by i.p. injection 20 minutes before the training phase reversed the scopolamine induced long term memory impairment in STPA determined 48 hours after training. Fluoxetine (1.54 mg/kg i.p. injection equimolar to 2.05 mg/kg NO-fluoxetine) did not have an effect on the 48 h retention phase. However, 5 of 12 animals receiving fluoxetine failed to learn the step-through passive avoidance task during the training phase, compared to zero in the fluoxetine group (failing animals were eliminated from the rest of the experiment).

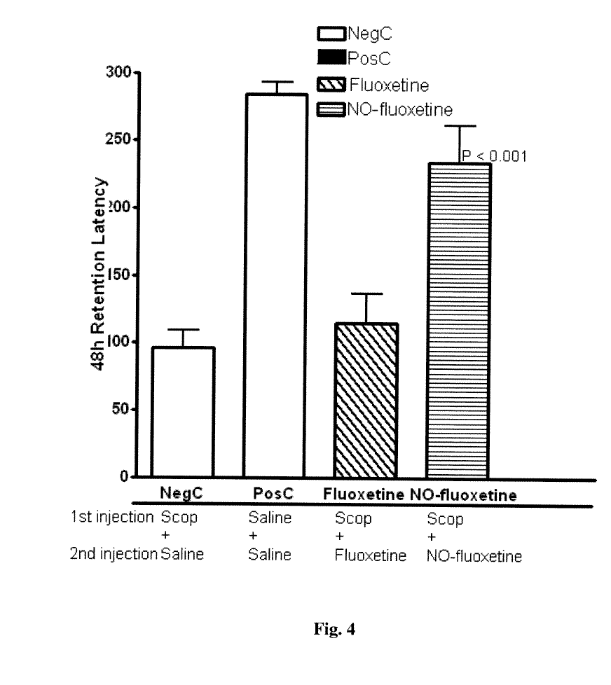

FIG. 4 shows the effect of NO-fluoxetine on the retention latency (48 h) in the STPA test. Mice received 1 mg/kg scopolamine by i.p. injections or water (positive control) 30 minutes before the training phase and 4.5 umole/kg of drug (fluoxetine or NO-fluoxetine) or water (pos and neg control) 20 minutes before the training phase. Values presented as mean±SEM, n=9-11 per group except fluoxetine group n=7 (5 out of 12 failed in the retention phase). The data were statistically evaluated by one way ANOVA, followed by Tukey post-hoc test.

In addition to fluoxetine, linker-fluoxetine, and NO-fluoxetine, other linker-SSRIs and NO-SSRIs were subjected to serotonin reuptake inhibition assays. The linker-SSRIs and NO-SSRIs tested were:

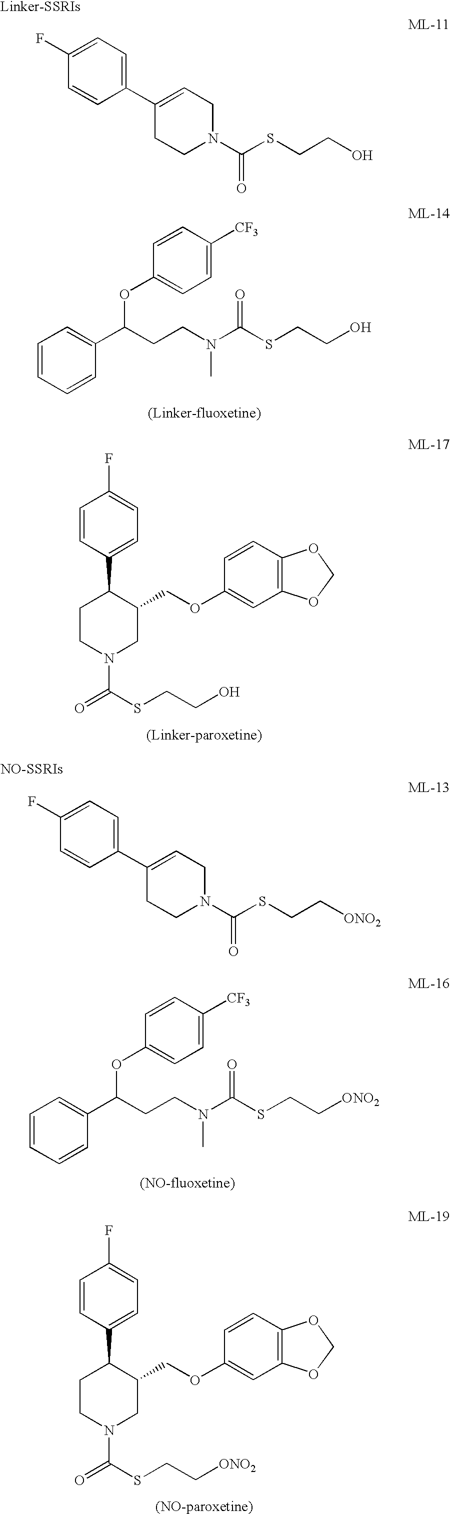

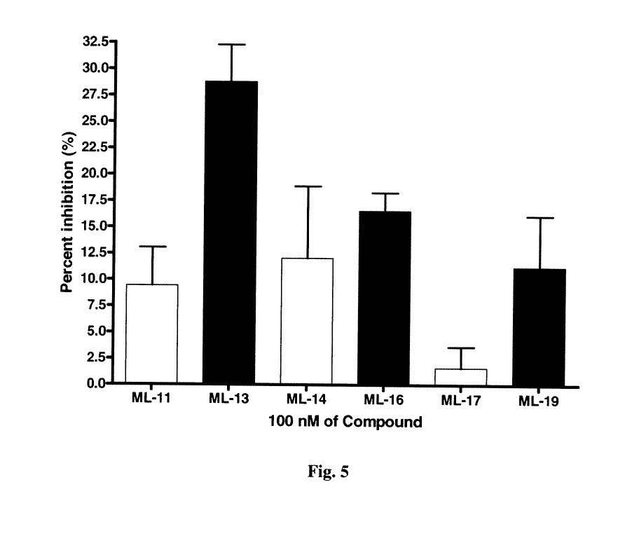

The results are summarized in FIG. 5. Like FIG. 2, FIG. 5 shows in vitro serotonin reuptake inhibitory activity of NO-SSRIs. Activity was determined by comparing H³-5HT transported into HEK cells stably transfected with human SERT and incubated with the drug for 10 minutes prior to H³-5HT exposure. 100% inhibition was obtained using 10 uM of fluoxetine. Extent of H³-5HT transported into the cell was determined by measuring the radioactivity of the cell lysates using a liquid scintillation counter.

The test results provided above show that NO-SSRI drugs have positive serotonin reuptake inhibitory activity at 100 nM concentrations. NO-fluoxetine was bioactivated in phosphate buffer, plasma, and rat liver microsomes to produce fluoxetine, and NO-fluoxetine released nitric oxide when incubated with GSH. In addition, NO-fluoxetine showed an antidepressant activity in the modified forced swim test. These result show that NO-fluoxetine releases both fluoxetine and nitric oxide in vivo, and produces an antidepressant activity in an in vitro model of serotonin reuptake activity and in an in vivo model of depression.

What is claimed:

1. A chimeric nitrate ester having a structural formula: 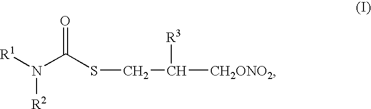
wherein R¹ is selected from the group consisting of C₁₋₆alkyl, C₃₋₈cycloalkyl, C₃₋₈heterocycloalkyl, aryl, and heteroaryl,
R² is selected from the group consisting of C₁₋₆alkyl, C₃₋₈cycloalkyl, C₃₋₈heterocycloalkyl, aryl, and heteroaryl, and 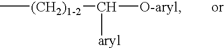
R¹ and R² are taken with the nitrogen atom to which they are attached to form a 5- or 6-membered ring, said ring substituted with an aryl or heteroaryl group and optionally with —(CH₂)_{1,2}—O-aryl, and
R³ is H or ONO₂,
or a pharmaceutically acceptable salt, prodrug, hydrate, or solvate thereof; or 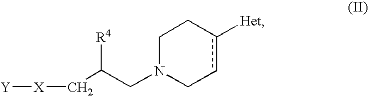
wherein X is null, O, or S,
R⁴ is —OH or —ONO,
Y is —CH₂aryl; —(CH₂)₁₋₂ONO₂, or 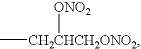
Het is heteroaryl, and 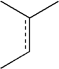
is a carbon-carbon single bond or a carbon-carbon double bond,
or a pharmaceutically acceptable salt, prodrug, hydrate, or solvate thereof; or 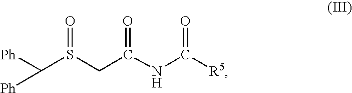
wherein Ph is phenyl, and 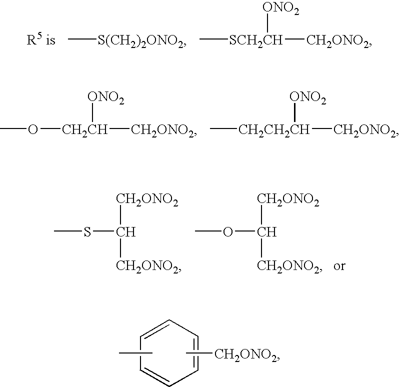
or a pharmaceutically acceptable salt, prodrug, hydrate, or solvate thereof.

2. The chimeric nitrate ester of claim 1 having a nitrate moiety positioned β, γ or δ to an organosulfur functionality.

3. The chimeric nitrate ester of claim 1 wherein
R¹ is C₁₋₆ alkyl, and preferably C₁₋₃ alkyl;
p is 0 or 1;
R² is 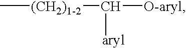
wherein the moiety —Oaryl optionally is substituted with a CF₃ group;
R³ is H or ONO₂; or
R¹ and R² are taken together with the nitrogen atom to which they are attached to form a 6-membered ring, wherein the 6-membered ring contains one substituent selected from (a) aryl optionally substituted with a halo and (b) heteroaryl, and further contains an optional —CH—O-aryl group, wherein said aryl group is selected from

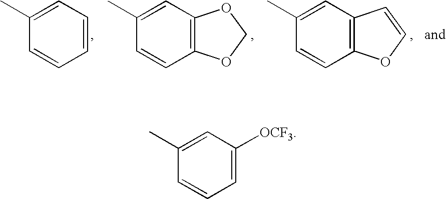

4. The chimeric nitrate ester of claim 1 wherein
X is null, O, or S;
Y is CH₂aryl, 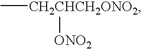
or —CH₂ONO₂;
R⁴ is —OH or —ONO₂;
Het is 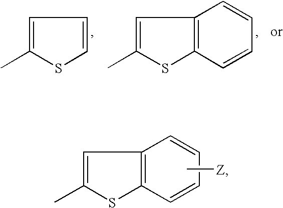
wherein Z is selected from the group consisting of halo, alkyl, alkoxy, CF₃, and OCF₃; and

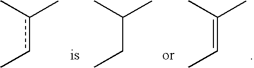

5. The chimeric nitrate ester of claim 1 selected from the group consisting of 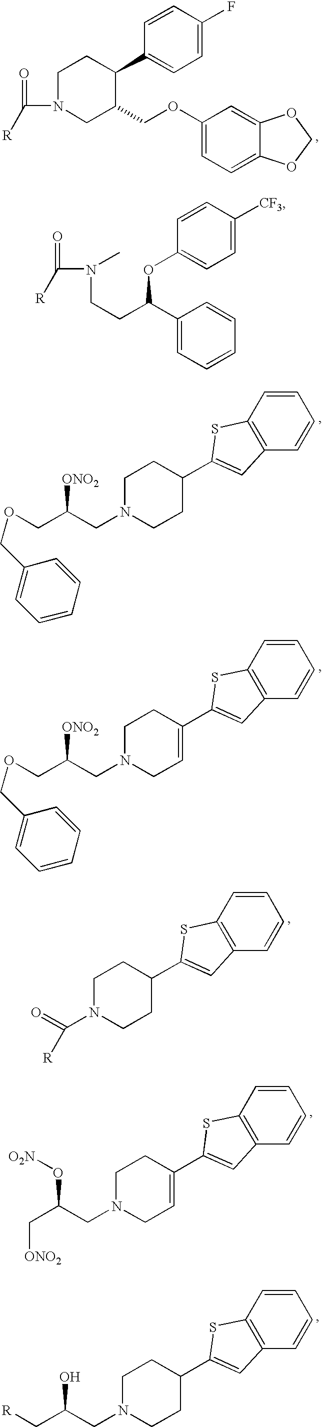 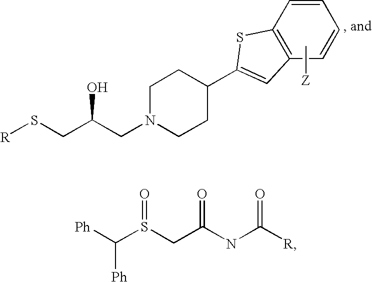
wherein R is selected from the group consisting of 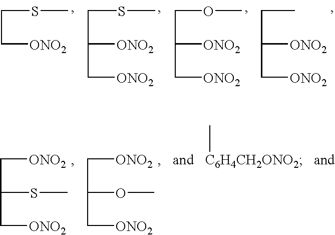
Z is selected from the group consisting of halo, alkyl, alkoxy, CF₃, and OCF₃.

6. The chimeric nitrate ester of claim 1 selected from the group consisting of

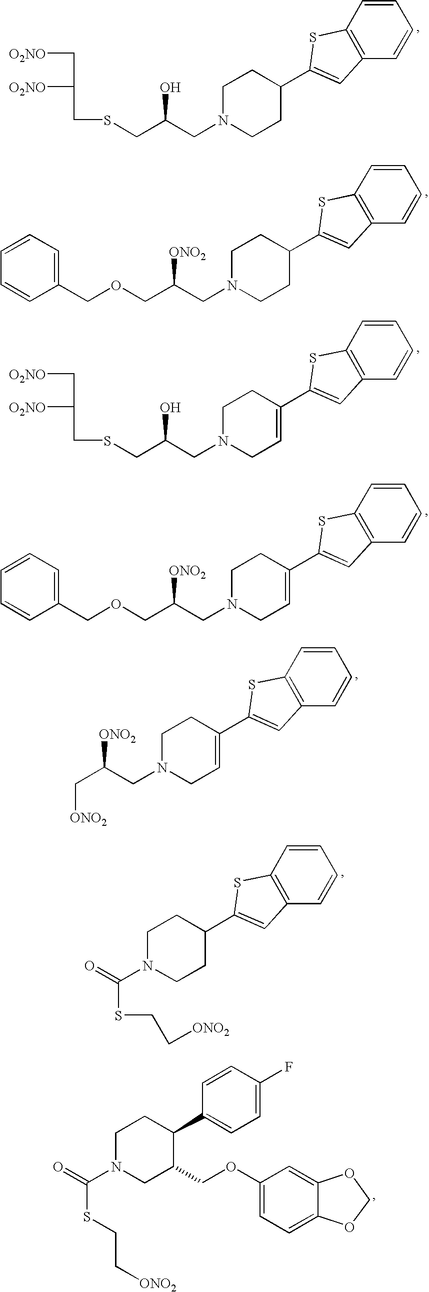

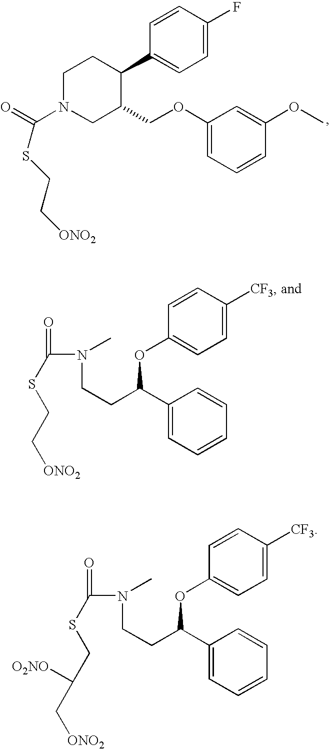

7. A method of treating a depression comprising administering a therapeutically effective amount of a nitrate ester of an antidepressant to an individual in need thereof.

8. The method of claim 7 wherein one or more symptom of a depression is treated.

9. The method of claim 7 wherein a therapeutic lag time for a full antidepressant therapeutic effect is about 2 to 7 days.

10. The method of claim 7 wherein the depression is a major depression, dysthymia, or both.

11. The method of claim 7 wherein the depression is a comorbidity, symptom, or prodromal of a neurodegenerative disorder, dementia, Parkinson's disease, epilepsy, or cancer.

12. The method of claim 7 wherein cognitive deficits associated with the depression are at least partially restored.

13. The method of claim 7 wherein the nitrate ester comprises a nitrated SSRI.

14. The method of claim 7 wherein the nitrate ester is selected from the group consisting of a nitrated fluoxetine, a nitrated paroxetine, a nitrated modafinil, a nitrated tianeptine, and mixtures thereof.

15. The method of claim 7 wherein the nitrate ester has a structural formula 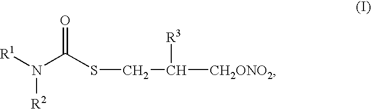
wherein R¹ is selected from the group consisting of C₁₋₆alkyl, C₃₋₈cycloalkyl, C₃₋₈heterocycloalkyl, aryl, and heteroaryl,
R² is selected from the group consisting of C₁₋₆alkyl, C₃₋₈cycloalkyl, C₃₋₈heterocycloalkyl, aryl, and heteroaryl, and 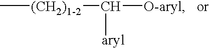
R¹ and R² are taken with the nitrogen atom to which they are attached to form a 5- or 6-membered ring, said ring substituted with an aryl or heteroaryl group and optionally with —(CH₂)_{1,2}—O-aryl, and
R³ is H or ONO₂,
or a pharmaceutically acceptable salt, prodrug, hydrate, or solvate thereof; or 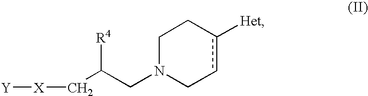
wherein X is null, O, or S,
R⁴ is —OH or —ONO₂,
Y is —CH₂aryl; —(CH₂)₁₋₂ONO₂, or 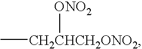
Het is heteroaryl, and 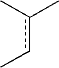
is a carbon-carbon single bond or a carbon-carbon double bond,
or a pharmaceutically acceptable salt, prodrug, hydrate, or solvate thereof, or 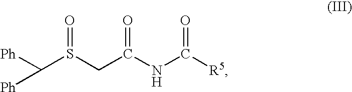
wherein Ph is phenyl, and 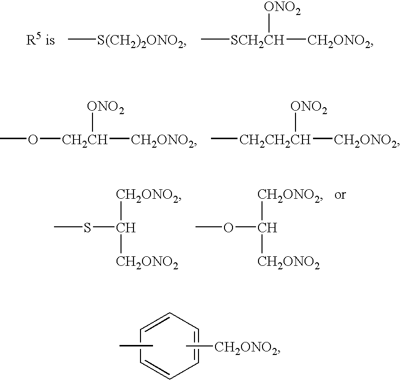
or a pharmaceutically acceptable salt, prodrug, hydrate, or solvate thereof.

16. The method of claim 7 wherein the nitrate ester has a structural formula 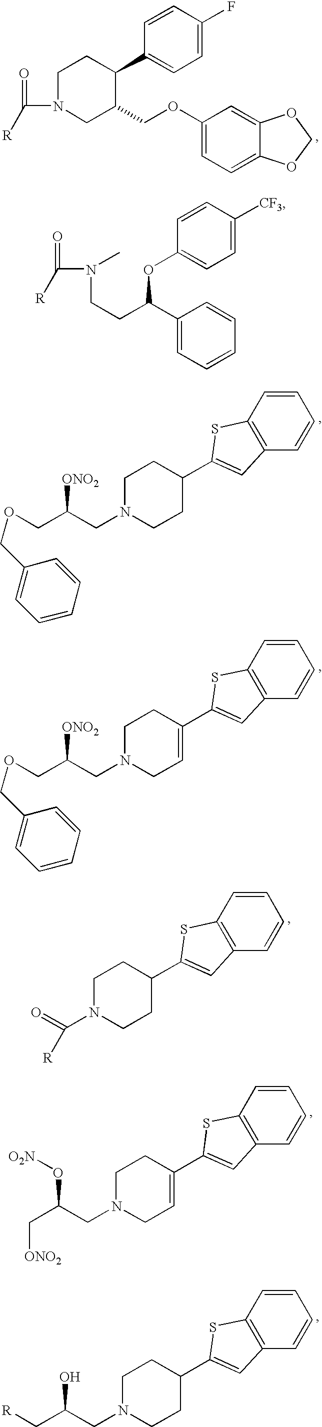 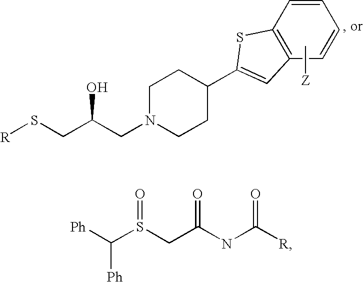
wherein R is selected from the group consisting of 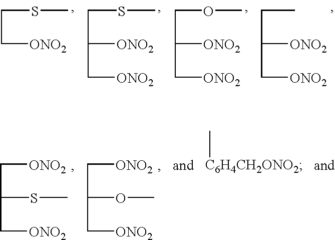
Z is selected from the group consisting of halo, alkyl, alkoxy, CF₃, and OCF₃.

17. The method of claim 7 wherein the nitrate ester has a structural formula

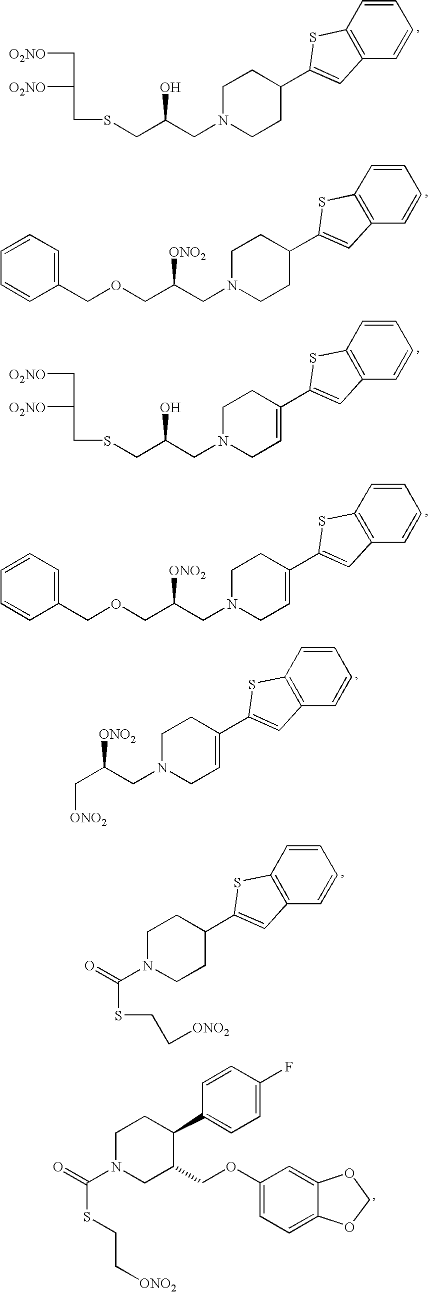

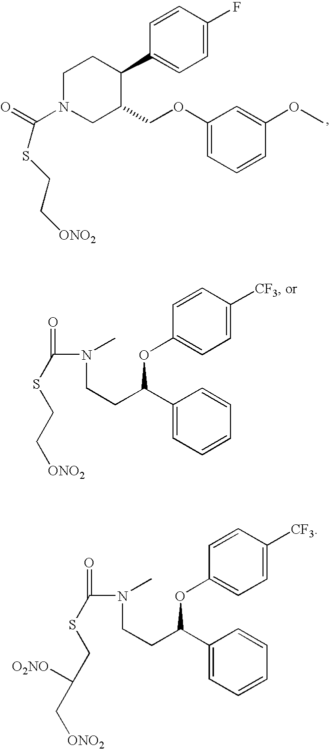

18. The method of claim 7 wherein the nitrate ester is capable of exhibiting antidepressant activity and donating nitric oxide in vivo.

19. The method of claim 7 wherein the nitrate ester has an NO mimetic moiety bound to an antidepressant moiety via a thiocarbamate, a carbamate, or an amide linkage.

20. A method of manufacturing a chimeric nitrate ester of structural formula (I) comprising reacting an amine R¹R²NH with an activated carbonyl compound in the presence of a mercaptan.

21. A method of manufacturing a chimeric nitrate ester of structural formula (I) comprising reacting a thiocarbamate derivative of an antidepressant drug with a brominating agent, then nitrating the resulting bromide compound.
